(19) United States
(12) Patent Application Publication
(10) Pub. No.: US 2021/0205253 A1
(43) Pub. Date: Jul. 8, 2021

(54) Use Of Stearic Acid For Preventing Or Treating Pulmonary Fibrosis

(71) Applicants: THE ASAN FOUNDATION, Seoul (KR); UNIVERSITY OF ULSAN FOUNDATION FOR INDUSTRY COOPERATION, Ulsan (KR)

(72) Inventors: Jin Woo SONG, Seoul (KR); Hyun Ju YOO, Seoul (KR); Jung Jin HWANG, Gyeonggi-do (KR)

(21) Appl. No.: 17/059,792

(22) PCT Filed: May 31, 2019
(86) PCT No.: PCT/KR2019/006584

§ 371 Date: Jan. 6, 2021

(30) Foreign Application Priority Data
May 31, 2018 (KR) 10-2018-0062704

(51) Int. Cl. A61K 31/20; A61K 9/00; A61K 31/7064; A61K 31/496; A61K 31/4418; A61P 11/00

(52) U.S. Cl. CPC A61K 31/20; A61K 9/0053; A61P 11/00; A61K 31/496; A61K 31/4418; A61K 31/7064

(57) ABSTRACT

The present invention relates to a composition for enhancing the sensitivity to a pulmonary fibrosis inhibitor, the composition comprising, as an active ingredient, stearic acid, a salt of the stearic acid or a prodrug of the stearic acid. In addition, the present invention relates to a pharmaceutical composition for preventing or treating pulmonary fibrosis, the composition comprising, as active ingredients: stearic acid, a salt of the stearic acid or a prodrug of the stearic acid; and a pulmonary fibrosis inhibitor. According to the present invention, a more excellent treatment effect may be induced by the co-administration of a conventional pulmonary fibrosis inhibitor and stearic acid, and by using stearic acid, the sensitivity to the conventional pulmonary fibrosis inhibitor may be enhanced, and an excellent treatment effect is expected to be achieved even for pulmonary fibrosis showing resistance to the conventional pulmonary fibrosis inhibitor.

### FIELD

The present invention relates to a use of a composition comprising, as active ingredients: stearic acid, a salt of the stearic acid or a prodrug of the stearic acid; and a pulmonary fibrosis inhibitor for preventing or treating pulmonary fibrosis.

### BACKGROUND

Fibrosis refers to a phenomenon in which a part of an organ hardens for some reason, and pulmonary fibrosis and hepatic fibrosis are considered as representative diseases. When chronic inflammation is repeated in the liver, the liver becomes cirrhotic, it hardens, and just as the liver loses its function, the lungs are also greatly affected by things other than inflammation and fibrosis occurs, and as the function of the lungs is gradually lost and oxygen supplied to the whole body is reduced, the function of other organs is also reduced. Among the characteristics of pulmonary fibrosis, the mechanism by which TGF-β changes pulmonary fibroblasts to the myofibroblast phenotype has been usually suggested, and tissue fibrosis, as defined by the excessive accumulation of the extracellular matrix (ECM), is a common pathological finding also observed in lung diseases due to various causes (European Respiratory Journal 2013-1271: 1207-120).

Various types of liver disease result in liver fibrosis, eventually leading to hepatic cirrhosis. Although the types of stimuli are different, such as hepatitis B, hepatitis C, alcohol and non-alcoholic liver disease, chronic damage to the liver results in an inflammatory response, and through the accumulation of the extracellular matrix, normal liver parenchyma is transformed into tissues such as regenerative nodules and scars, resulting in fibrosis. Previously, hepatic fibrosis and cirrhosis were known as irreversible reactions, but recently there are many reports that cirrhosis can also ameliorated when the cause of liver injury is eliminated or treated.

In contrast, pulmonary fibrosis found in diseases such as idiopathic pulmonary fibrosis is caused by excessive accumulation of the extracellular matrix due to impaired normal wound healing processes. That is, unlike liver fibrosis and cirrhosis caused by an inflammatory response, fibrosis occurs even if there is no confirmed inflammatory response in pulmonary fibrosis. Currently, there are two FDA-approved therapeutic agents for idiopathic pulmonary fibrosis, pirfenidone and nintedanib, and these drugs have been confirmed to slow the progression of pulmonary fibrosis, but there is no evidence that the drugs will ameliorate pulmonary fibrosis, and therapeutic agents which interrupt or ameliorate the progression of the disease itself have not yet been commercialized. Further, in the case of pirfenidone and nintedanib, 90% or more of the patients who took the drug experienced side effects, and 20 to 30% of the patients discontinued use of the drug after one year. Therefore, there is an urgent need for developing a drug with few side effects while simultaneously interrupting or ameliorating the progression of pulmonary fibrosis.

The matters described as the aforementioned background art are only for the purpose of improving the understanding of the background of the present invention, and should not be taken as acknowledging that they correspond to the related art already known to those skilled in the art.

### SUMMARY

### Technical Problem

As a result of intensive efforts to overcome the limitations and side effects of existing pulmonary fibrosis inhibitors as therapeutic agents, the present inventors confirmed that when stearic acid, which is an endogenous fatty acid, was co-administered in vivo with an existing pulmonary fibrosis inhibitor, existing side effects such as a reduction in body weight could be ameliorated and various fibrosis indices could be substantially improved, thereby completing the present invention.

Therefore, an object of the present invention is to provide a composition for enhancing the sensitivity to a pulmonary fibrosis inhibitor, the composition comprising, as an active ingredient, stearic acid, a salt of the stearic acid or a prodrug of the stearic acid.

Another object of the present invention is to provide a pharmaceutical composition for preventing or treating pulmonary fibrosis, the composition comprising, as active ingredients: stearic acid, a salt of the stearic acid or a prodrug of the stearic acid; and a pulmonary fibrosis inhibitor.

Still another object of the present invention is to provide a food composition for preventing or ameliorating pulmonary fibrosis, the composition comprising, as active ingredients: stearic acid, a salt of the stearic acid or a prodrug of the stearic acid; and a pulmonary fibrosis inhibitor.

Yet another object of the present invention is to provide a therapeutic aid for pulmonary fibrosis having resistance to a pulmonary fibrosis inhibitor, the aid comprising, as an active ingredient, stearic acid, a salt of the stearic acid or a prodrug of the stearic acid.

Yet another object of the present invention is to provide a pharmaceutical composition for inhibiting side effects by a pulmonary fibrosis inhibitor, the composition comprising, as an active ingredient, stearic acid, a salt of the stearic acid or a prodrug of the stearic acid.

Yet another object of the present invention is to provide a method for providing information on whether or not to co-administer stearic acid, a salt of the stearic acid or a prodrug of the stearic acid.

However, technical problems to be achieved by the present invention are not limited to the aforementioned problems, and other problems that are not mentioned may be clearly understood by those skilled in the art from the following description.

### Technical Solution

To achieve the objects of the present invention, the present invention provides a composition for enhancing the sensitivity to a pulmonary fibrosis inhibitor, the composition comprising, as an active ingredient, stearic acid, a salt of the stearic acid or a prodrug of the stearic acid.

As an exemplary embodiment of the present invention, the pulmonary fibrosis inhibitor may be selected from the group consisting of pirfenidone, nintedanib, trimethoprim/sulfamethoxazole (co-trimoxazole), a recombinant human pentraxin-2 protein (PRM-151), romilkimab (SAR156597), pamrevlumab, BG00011, treprostinil, TD139, CC-90001, 2-((2-ethyl-6-(4-(2-(3-hydroxyazetidin-1-yl)-2-oxoethyl)piperazin-1-yl)-8-methylimidazo[1,2-a]pyridin-3-yl)(methyl)amino)-4-(4-fluorophenyl)thiazole-5-carbonitrile) (GLPG1690), losartan, tetrathiomolybdate, lebrikizumab, zileuton, nandrolone decanoate, sirolimus, everolimus, vismodegib, fresolimumab, omipalisib (GSK2126458), (3S)-3-[3-(3,5-dimethyl-1H-pyrazol-1-yl)phenyl]-4-{(3S)-3-[2-(5,6,7,8-tetrahydro-1,8-naphthyridin-2-yl)ethyl]-1-pyrrolidinyl}butanoic acid (GSK3008348), rituximab, octreotide, 2-[3-[4-(1H-indazol-5-ylamino)-2-quinazolinyl]phenoxy]-N-(1-methylethyl)-acetamide (KD025), tipelukast (MN-001), BBT-877, OLX201, DWN12088, and a salt thereof.

As another exemplary embodiment of the present invention, the pulmonary fibrosis may be idiopathic pulmonary fibrosis (IPF).

As still another exemplary embodiment of the present invention, the above-mentioned pulmonary fibrosis may have an increase in activation of pulmonary fibroblasts and an increase in loss of pulmonary epithelial cells due to TGF-beta compared to the case where there is no pulmonary fibrosis.

As yet another exemplary embodiment of the present invention, the pulmonary fibrosis may have increases in both of fibrosis markers, collagen 1 (COL-1) and alpha-smooth muscle actin (α-SMA), in pulmonary fibroblasts compared to the case where there is no pulmonary fibrosis.

Further, the present invention provides a pharmaceutical composition for preventing or treating pulmonary fibrosis, the composition comprising, as active ingredients: (i) stearic acid, a salt of the stearic acid or a prodrug of the stearic acid; and (ii) a pulmonary fibrosis inhibitor.

As an exemplary embodiment of the present invention, stearic acid, a salt of the stearic acid, or a prodrug of the stearic acid:pirfenidone may be included at a molar concentration ratio of 1:0.5 to 1:25 in the composition.

As another exemplary embodiment of the present invention, stearic acid, a salt of the stearic acid, or a prodrug of the stearic acid:nintedanib may be included at a molar concentration ratio of 1:0.01 to 1:5 in the composition.

In addition, the present invention provides a therapeutic aid for pulmonary fibrosis having resistance to a pulmonary fibrosis inhibitor, the aid comprising, as an active ingredient, stearic acid, a salt of the stearic acid or a prodrug of the stearic acid.

Furthermore, the present invention provides a pharmaceutical composition for inhibiting side effects by a pulmonary fibrosis inhibititor, the composition comprising, as an active ingredient, stearic acid, a salt of the stearic acid or a prodrug of the stearic acid.

Further, the present invention provides a method for enhancing the sensitivity to a pulmonary fibrosis inhibitor, the method comprising: administering, to an individual, a composition comprising, as an active ingredient, stearic acid, a salt of the stearic acid or a prodrug of the stearic acid.

In addition, the present invention provides a method for preventing or treating pulmonary fibrosis, the method comprising: administering, to an individual, (i) stearic acid, a salt of the stearic acid or a prodrug of the stearic acid; and (ii) a pulmonary fibrosis inhibitor.

Furthermore, the present invention provides a method for inhibiting side effects by a pulmonary fibrosis inhibitor, the method comprising: administering, to an individual, a composition comprising, as an active ingredient, stearic acid, a salt of the stearic acid or a prodrug of the stearic acid.

### Advantageous Effects

The present inventors confirmed an anti-fibrotic effect of stearic acid as a diagnostic marker and therapeutic target for pulmonary fibrosis, and confirmed that a more excellent anti-fibrotic effect occurred compared to the above inhibitor alone by co-administering a pulmonary fibrosis inhibitor pirfenidone or nintedanib with stearic acid based on the anti-fibrotic effect. Thus, according to the present invention, a more excellent treatment effect can be induced by the co-administration of a conventional pulmonary fibrosis inhibitor and stearic acid, and by using stearic acid, the sensitivity to the conventional pulmonary fibrosis inhibitor can be enhanced, and drug side effects occurring in a patient can be reduced, and an excellent treatment effect is expected to be achieved even for pulmonary fibrosis showing resistance to the conventional pulmonary fibrosis inhibitor.

### BRIEF DESCRIPTION OF DRAWINGS

FIG. 1 illustrates the results of quantification of free fatty acids in human lung tissue (Normal: lung tissues derived from normal group patients, n=10; IPF: lung tissues derived from patients with idiopathic pulmonary fibrosis, n=10).

FIG. 2 illustrates the results of exhibiting a value obtained by dividing the amount of stearic acid by the amount of free fatty acids having 14 to 18 carbon atoms based on the quantification result of free fatty acids in a human lung tissue of FIG. 1 (Normal: lung tissues derived from normal group patients, n=10; IPF: lung tissues derived from patients with idiopathic pulmonary fibrosis, n=10).

FIG. 3A illustrates the results of exhibiting the effect of stearic acid on fibroblast activation by TGF-β when cells are treated with TGF-β and stearic acid (SA) together.

FIG. 3B illustrates the results of exhibiting the effect of stearic acid on the loss of epithelial cells by TGF-β when cells are treated with TGF-β and stearic acid (SA) together.

FIG. 4A illustrates the results of exhibiting the change in collagen 1 (Collagen 1/ACTIN), which is a marker of fibrosis in fibroblasts, caused by stearic acid, as a relative value to a control (CTL) (here, Collagen 1/ACTIN indicates a value obtained by correcting the amount of Collagen 1 protein with actin, which is an intracellular control protein).

FIG. 4B illustrates the results of exhibiting the change in α-SMA (α-SMA/ACTIN), which is a marker of fibrosis in fibroblasts, caused by stearic acid, as a relative value to a control (CTL) (here, α-SMA/ACTIN indicates a value obtained by correcting the amount of α-SMA protein with actin, which is an intracellular control protein).

FIG. 5A illustrates the results exhibiting the effect on fibroblast activation when cells are treated with palm itic acid (PA) at various concentrations.

FIG. 5B illustrates the results exhibiting the effect on the loss of epithelial cells when cells are treated with palm itic acid (PA) at various concentrations.

FIG. 6A illustrates the results exhibiting the change in collagen 1 (Collagen 1/ACTIN), which is a marker of fibrosis in fibroblasts, caused by palm itic acid (PA), as a relative value to a control (CTL).

FIG. 6B illustrates the results exhibiting the change in α-SMA(α-SMA/ACTIN), which is a marker of fibrosis in fibroblasts, caused by palmitic acid (PA), as a relative value to a control (CTL).

FIG. 7A illustrates the results exhibiting the change in collagen 1 (Collagen 1/ACTIN), which is a marker of fibrosis in pulmonary fibroblasts, as a relative value to a control (CTL) when pulmonary fibroblasts are treated with TGF-β, treated with palmitic acid, treated with stearic acid, co-treated with TGF-β and stearic acid and co-treated with palmitic acid and stearic acid (CTL: control, TGF-b: TGF-β 5 ng/mL treatment group, PA: palmitic acid 10 uM/mL treatment group, SA: stearic acid 40 uM/mL treatment group, TGF-b+SA: TGF-β 5 ng/mL+stearic acid 40 uM/mL combined treatment group, PA+SA: palmitic acid 10 uM/mL+stearic acid 40 uM/mL combined treatment group).

FIG. 7B illustrates the results exhibiting the change in α-SMA (α-SMA/ACTIN), which is a marker of fibrosis in pulmonary fibroblasts, as a relative value to a control (CTL) when pulmonary fibroblasts are treated with TGF-β, treated with palmitic acid, and treated with stearic acid, co-treated with TGF-β and stearic acid and co-treated with palm itic acid and stearic acid under the same conditions as in FIG. 7A.

FIG. 8A illustrates the change in collagen 1 (Collagen 1/ACTIN), which is a marker of fibrosis in pulmonary fibroblasts, as a relative value to a control (CTL) when pulmonary fibroblasts are treated with TGF-β, treated with oleic acid (OA), and treated with stearic acid, co-treated with TGF-β and stearic acid and co-treated with oleic acid and stearic acid (CTL: control, TGF-b: TGF-β 5 ng/mL treatment group, OA: oleic acid 40 uM/mL treatment group, SA: stearic acid 40 uM/mL treatment group, TGF-b+SA: TGF-β 5 ng/mL+stearic acid 40 uM/mL combined treatment group, OA+SA: oleic acid 40 uM/mL+stearic acid 40 uM/mL combined treatment group).

FIG. 8B illustrates the results exhibiting the change in α-SMA (α-SMA/ACTIN), which is a marker of fibrosis in pulmonary fibroblasts, as a relative value to a control (CTL) when pulmonary fibroblasts are treated with TGF-β, treated with oleic acid (OA), treated with stearic acid, co-treated with TGF-β and stearic acid and co-treated with oleic acid and stearic acid under the same conditions as in FIG. 8A.

FIG. 9A illustrates the results of measuring the change in body weights of mice after administration of stearic acid in a pulmonary fibrosis animal model induced by bleomycin (Normal control (Con, n=4), bleomycin single administration group (Bleo, n=5), stearic acid administration group (SA, n=4), bleomycin+stearic acid administration group (Bleo+SA, n=6))(**p<0.01 and *p<0.05 are p values when compared with the control. # p<0.05 is a p value when compared with the bleomycin treatment group).

FIG. 9B illustrates the results of lung tissue staining (H&E) of mice after administration of stearic acid in the same pulmonary fibrosis animal model as in FIG. 9A.

FIG. 9C illustrates the results of measuring and comparing the content of hydroxyproline after administration of stearic acid in the same pulmonary fibrosis animal model as in FIG. 9A.

FIG. 9D illustrates the results of measuring the expression level of α-SMA in lung tissues after administration of stearic acid in the same fibrosis pulmonary fibrosis animal model as in FIG. 9A.

FIG. 9E illustrates the results of measuring the expression level of p-Smad2/3 in lung tissues after administration of stearic acid in the same fibrosis pulmonary fibrosis animal model as in FIG. 9A.

FIG. 9F illustrates the results of measuring the change in TGF-β1 in serum after administration of stearic acid in the same fibrosis pulmonary fibrosis animal model as in FIG. 9A.

FIG. 10A illustrates the results exhibiting the effect of inhibiting the expression of Collagen 1 and α-SMA, which are fibrosis markers, according to an increase in the treatment concentration of stearic acid in human primary fibroblasts by immunoblotting.

FIG. 10B illustrates the results of comparing the effects of inhibiting the expression of Collagen 1 and α-SMA, which are fibrosis markers, according to an increase in the treatment concentration of stearic acid in human primary fibroblasts via Fold induction.

FIG. 10C illustrates the results exhibiting the inhibitory effect on Collagen 1 and α-SMA, which are fibrosis markers, according to the treatment with stearic acid in the primary fibroblasts obtained from 4 patients.

FIG. 10D illustrates the results exhibiting the inhibitory effect on Collagen 1 and α-SMA, which are fibrosis markers, according to the treatment of stearic acid against TGF-β stimulation by immunoblotting.

FIG. 10E illustrates the results of comparing the inhibitory effect on Collagen 1 and α-SMA, which are fibrosis markers, according to the treatment with stearic acid against TGF-β stimulation via Fold induction (*p<0.05 is a p value when compared with the control, and # p<0.05 is a p value when compared with the bleomycin treatment group).

FIG. 11A illustrates the results of measuring the change in the expression of E-cadherin caused by stearic acid in epithelial cells by immunoblotting.

FIG. 11B illustrates the change in the expression of E-cadherin (E-cadherin/Actin) caused by stearic acid in epithelial cells as a relative value to the control (CTL)(*p<0.05 is a p value when compared with the control, and # p<0.05 is a p value when compared with the bleomycin treatment group).

FIG. 12A illustrates the results of measuring the expression of p-Smad2/3 and Smad7 proteins according to the treatment with stearic acid in fibroblasts by immunoblotting.

FIG. 12B illustrates the results of comparing the expression of p-Smad2/3 and Smad7 proteins according to the treatment with stearic acid in fibroblasts via Fold induction.

FIG. 12C illustrates the results of measuring the change in ROS after treatment with stearic acid and/or TGF-β1.

FIG. 12D illustrates the results of measuring the change in the expression of p-Smad2/3 according to the treatment with TGF-β1 and/or an antioxidant (NAC).

FIG. 13 illustrates, as the results of confirming the anti-fibrotic effect according to the combined treatment with stearic acid and pirfenidone in human-derived primary fibroblasts, the results of treating the cells with TGF-β (5 ng/ml), stearic acid (40 μM), and/or pirfenidone (400 or 800 μM), and then measuring the expression levels of collagen 1 (COL-1) and α-SMA, which are fibrosis markers and quantitatively analyzing the inhibitory efficiency of each of collagen 1 (COL-1) and α-SMA.(TGF: TGF-β single treatment group, TGF+PIR: TGF-β and pirfenidone treatment group, TGF+Combi: TGF-β and pirfenidone+stearic acid combined treatment group).

FIG. 14 illustrates the results of confirming the anti-fibrotic effect on MRC-5, which is a human fibroblast cell line, according to the combined treatment with stearic acid and pirfenidone in the same manner as in FIG. 13.

FIG. 15 illustrates, as the results of confirming the anti-fibrotic effect according to the combined treatment with stearic acid and pirfenidone in a human pulmonary epithelial cell line BEAS-2B, the results of treating the cells with TGF-β (5 ng/ml), stearic acid (40 μM), and/or pirfenidone (800 μM), and then measuring the expression of fibronectin with a marker of EMT, which is one of the pulmonary fibrosis indices, and quantitatively analyzing the inhibitory efficiency thereof (TGF: TGF-β single treatment group, TGF+PIR: TGF-β and pirfenidone treatment group, TGF+Combi: TGF-β and pirfenidone+stearic acid combined treatment group).

FIG. 16A illustrates the results exhibited by measuring the change in body weight after administering each of stearic acid and pirfenidone or co-administering stearic acid and pirfenidone and quantitatively analyzing the result on day 21 after administration in order to confirm the anti-fibrotic effect of the combined administration of stearic acid and pirfenidone in an animal model in which pulmonary fibrosis was induced by administration of bleomycin (Ctrl: normal control, Bleo: bleomycin single administration group, Bleo+PIR(P): bleomycin and pirfenidone administration group, Bleo+SA: bleomycin and stearic acid administration group, Bleo+P+SA (or Bleo+combi): bleomycin and pirfenidone+stearic acid combined administration group).

FIG. 16B illustrates the results of measuring the hydroxyproline levels in the above animal model to which stearic acid and/or pirfenidone were/was administered and quantitatively analyzing and comparing the hydroxyproline levels (Ctrl: normal control, Bleo: bleomycin single administration group, Bleo+PIR: bleomycin and pirfenidone administration group, Bleo+SA: bleomycin and stearic acid administration group, Bleo+P+S (or Bleo+combi): bleomycin and pirfenidone+stearic acid combined administration group).

FIG. 17A illustrates the results of treating human-derived primary fibroblasts with TGF-β (5 ng/ml), stearic acid (40 μM) and/or nintedanib (1.5 or 2 μM) and measuring the expression levels of collagen 1 (COL-1) and α-SMA, which are fibrosis markers, in order to confirm the anti-fibrotic effect according to the combinatory treatment of stearic acid and nintedanib in human-derived primary fibroblasts.

FIG. 17B illustrates the results of treating human-derived primary fibroblasts with TGF-β (5 ng/ml), stearic acid (40 μM) and/or nintedanib (2 μM), measuring the expression levels of collagen 1 (COL-1) and α-SMA, and quantitatively analyzing the inhibitory efficiency of COL-1 (TGF: TGF-β single treatment group, TGF+NIN: TGF-β and nintedanib treatment group, TGF+Combi: TGF-β and nintedanib+stearic acid combinatory treatment group).

### DETAILED DESCRIPTION

The present inventors have made efforts to seek a method capable of overcoming limitations (which slow the progression of fibrosis but have no substantial therapeutic effect) as a therapeutic agent of an existing pulmonary fibrosis inhibitor and various side effects such as a reduction in body weight, and as a result, have discovered the possibility of overcoming the limitations of the aforementioned existing therapeutic agents when administering stearic acid, which is an endogenous fatty acid, in vivo.

As used in the present invention, the term “pulmonary fibrosis” can be used to mean any disease in which a lung tissue is fibrotic, and thus induces a respiratory disorder, but may be, for example, idiopathic pulmonary fibrosis (IPF) characterized by pulmonary fibrosis, an interstitial lung disease such as idiopathic interstitial pneumonia and a connective tissue disease associated interstitial lung disease, or hypersensitivity pneumonitis, and more preferably idiopathic pulmonary fibrosis (IPF).

According to a preferred exemplary embodiment of the present invention, the pulmonary fibrosis has an increase in activation of pulmonary fibroblasts and an increase in loss of pulmonary epithelial cells caused by TGF-β, or an increase in collagen 1 (COL-1) and α-SMA in pulmonary fibroblasts, compared to the case where there is no pulmonary fibrosis, and the aforementioned characteristics may be exhibited together.

The idiopathic pulmonary fibrosis is also called idiopathic pulmonary fibrosis, and refers to a disease which causes a structural change in lung tissue due to an increase in deposition of fibroblasts and collagen caused by repeated damage to the alveolar wall and abnormalities in the wound recovery process without known causes, and gradually aggravates pulmonary dysfunction, and as a result, leads to death in cases where the symptoms are severe.

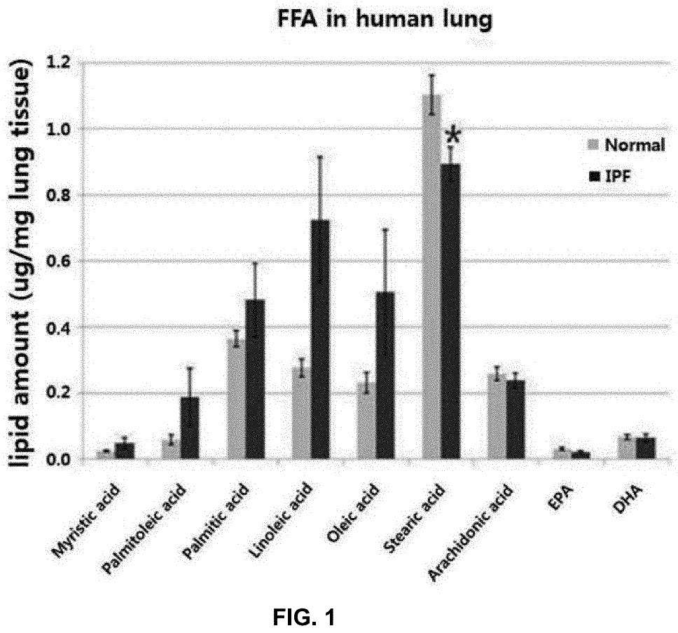

In an exemplary embodiment of the present invention, as can be seen in FIG. 1, it was confirmed that the contents of saturated or unsaturated free fatty acids having 16 to 18 carbon atoms (for example, palmitoleic acid, palmitic acid, linolenic acid, oleic acid, stearic acid, and the like), for example, stearic acid, in fibrotic tissues exhibited a remarkable difference compared to those in normal tissues. In particular, it was confirmed that the content of stearic acid in fibrotic tissues was significantly reduced compared to normal tissues, and the contents of linolenic acid and oleic acid, preferably, palmitoleic acid, palmitic acid, linolenic acid, and oleic acid in fibrotic tissues were increased compared to those in normal tissues.

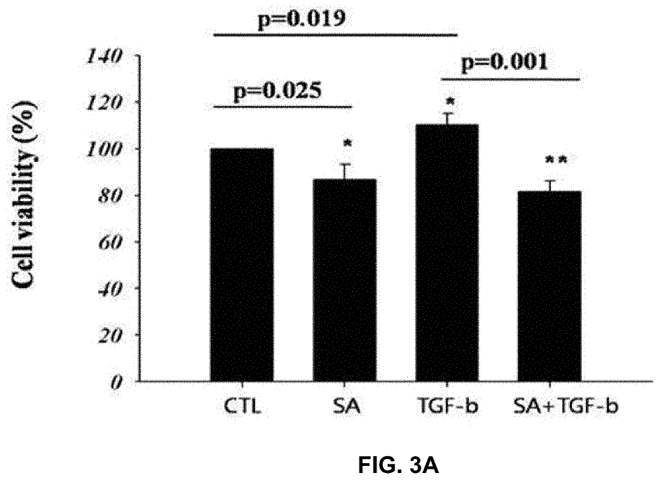

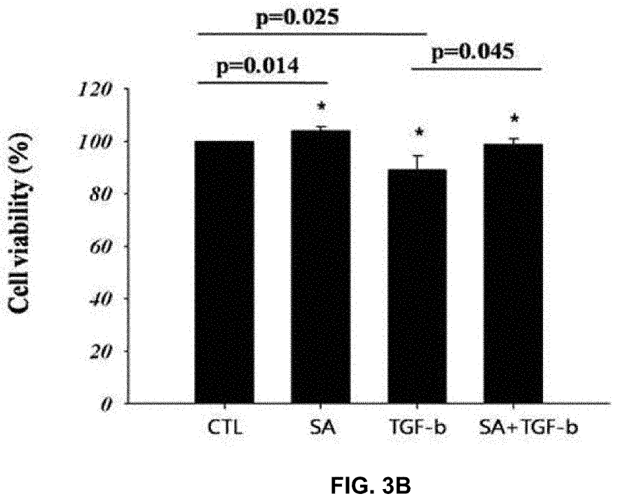

Furthermore, the present inventors focused on a reduction (deficiency) in the content of stearic acid in fibrotic tissues as described above, and confirmed that a fibrosis therapeutic effect could be obtained by administering stearic acid (see FIGS. 3 to 12). Therefore, based on these results, the present inventors propose the use of stearic acid as a therapeutic agent for pulmonary fibrosis, for example, idiopathic pulmonary fibrosis.

Specifically, the present invention provides a composition for treating, ameliorating, and/or preventing fibrosis, the composition comprising, as an active ingredient, stearic acid, a salt of the stearic acid or a prodrug of the stearic acid. The active ingredient means an ingredient that exerts a desired effect, for example, an effect for treating, ameliorating and/or preventing fibrosis.

In the present invention, stearic acid may include an octadecanoic acid with the formula C₁₇H₃₅CO₂H having an 18 carbon chain and a derivative or prodrug in which one or more of the hydrogen atoms of the above Formula are substituted.

As used herein, the term prodrug refers to a drug whose physical and chemical properties are adjusted by chemically changing a drug, and means that although the prodrug does not show physiological activity by itself, the prodrug after administration is changed into an original drug chemically or by the action of an enzyme in the body to exert its medicinal effect, and the prodrug in the present invention may include a prodrug of stearic acid capable of exhibiting the same or very similar effect as stearic acid in the body.

The stearic acid may be prepared as a derivative or prodrug by introducing a substituent by various methods known in the art according to the intended use, and is understood to be included in the scope of the present invention. Examples of the derivative or prodrug include methyl stearate, ethyl stearate, butyl stearate, vinyl stearate, stearyl stearate, triethanolamine stearate, glyceryl tr(stearate), isopropyl isostearate, ethylene glycol monostearate, propylene glycol monostearate, glycerol monostearate, PEGylated stearate, L-ascorbic acid 6-stearate, 2-butoxyethyl stearate, 4-nitrophenyl stearate, lauryl stearate, isooctyl stearate, cholesteryl stearate, and the like, but are not limited thereto.

According to an aspect of the present invention, the present invention provides a composition for enhancing the sensitivity to a pulmonary fibrosis inhibitor, the composition comprising, as an active ingredient, stearic acid, a salt of the stearic acid or a prodrug of the stearic acid.

In the present invention, the term pulmonary fibrosis inhibitor is used to mean including a therapeutic agent for pulmonary fibrosis, and refers to a drug that interrupts, delays, prevents, ameliorates or treats the progression of pulmonary fibrosis, and may be preferably selected from the group consisting of pirfenidone, nintedanib, trimethoprim/sulfamethoxazole (co-trimoxazole), a recombinant human pentraxin-2 protein (PRM-151), romilkimab (SAR156597), pamrevlumab, BG00011, treprostinil, TD139, CC-90001, 2-((2-ethyl-6-(4-(2-(3-hydroxyazetidin-1-yl)-2-oxoethyl)piperazin-1-yl)-8-methylimidazo[1,2-a]pyridin-3-yl)(methyl)amino)-4-(4-fluorophenyl)thiazole-5-carbonitrile) (GLPG1690), losartan, tetrathiomolybdate, lebrikizumab, zileuton, nandrolone decanoate, sirolimus, everolimus, vismodegib, fresolimumab, omipalisib (GSK2126458), (3S)-3-[3-(3,5-dimethyl-1H-pyrazol-1-yl)phenyl]-4-{(3S)-3-[2-(5,6,7,8-tetrahydro-1,8-naphthyridin-2-ypethyl]-1-pyrrolidinyl}butanoic acid (GSK3008348), rituximab, octreotide, 2-[3-[4-(1H-indazol-5-ylamino)-2-quinazolinyl]phenoxy]-N-(1-methylethyl)-acetamide (KD025), tipelukast (MN-001), BBT-877, OLX201, DWN12088, and a salt thereof.

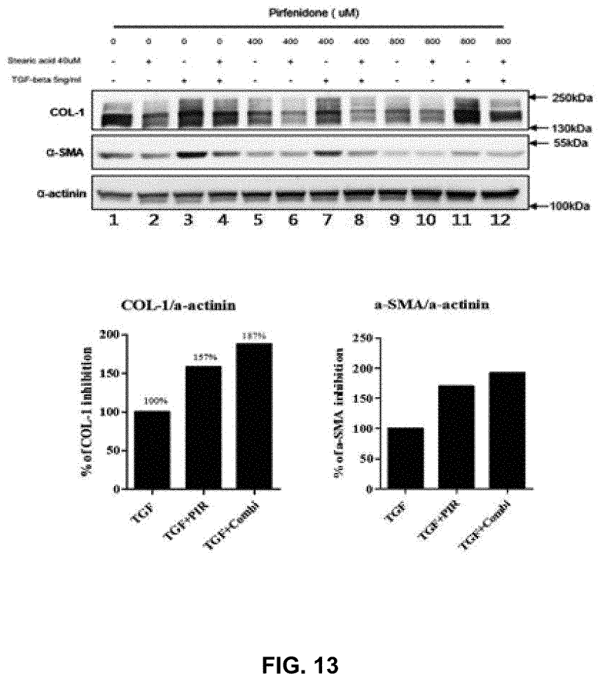

According to an exemplary embodiment of the present invention, the present inventors experimentally confirmed that by using an animal model in which a fibrosis marker index (COL-1 and/or α-SMA) was inhibited, EMT was inhibited, and/or pulmonary fibrosis was induced, the anti-fibrotic effect was remarkably increased when stearic acid was co-administered compared to when cells were treated with an existing pulmonary fibrosis inhibitor, for example, pirfenidone or nintedanib, alone (see FIGS. 13 to 17).

Therefore, according to another aspect of the present invention, the present invention provides a pharmaceutical composition for preventing or treating pulmonary fibrosis, the composition comprising, as active ingredients: (i) stearic acid, a salt of the stearic acid or a prodrug of the stearic acid; and (ii) a pulmonary fibrosis inhibitor.

In the present invention, stearic acid, a salt of the stearic acid or a prodrug of the stearic acid:pirfenidone may be included at a molar concentration ratio of 1:0.5 to 1:25, preferably 1:1 to 1:23, more preferably 1:5 to 1:22, even more preferably 1:8 to 1:21, and most preferably 1:10 to 1:20, in the composition.

In the present invention, stearic acid, a salt of the stearic acid or a prodrug of the stearic acid:nintedanib may be included at a molar concentration ratio of 1:0.01 to 1:5, preferably 1:0.02 to 1:1, more preferably 1:0.025 to 1:0.5, even more preferably 1:0.03 to 1:0.1, and most preferably 1:0.03 to 1:0.05, in the composition.

As used herein, the term “prevention” refers to all actions that suppress pulmonary fibrosis or delay the onset of the pulmonary fibrosis by administering the pharmaceutical composition according to the present invention.

As used herein, the term “treatment” refers to all actions that ameliorate or beneficially change symptoms caused by pulmonary fibrosis by administering the pharmaceutical composition according to the present invention.

As used herein the term salt or “pharmaceutically acceptable salt” refers to a formation of a compound which does not induce serious irritation in the organism to which the compound is administered and does not impair the biological activity and physical properties of the compound. The pharmaceutical salt may be obtained by reacting the compound of the present invention with an inorganic acid such as hydrochloric acid, bromic acid, sulfuric acid, nitric acid, and phosphoric acid, a sulfonic acid such as methanesulfonic acid, ethanesulfonic acid, and p-toluenesulfonic acid, and an organic carbonic acid such as tartaric acid, formic acid, citric acid, acetic acid, trichloroacetic acid, trifluoroacetic acid, capric acid, isobutanoic acid, malonic acid, succinic acid, phthalic acid, gluconic acid, benzoic acid, lactic acid, fumaric acid, maleic acid, and salicyclic acid. Further, the pharmaceutical salt may also be obtained by reacting the compound of the present invention with a base to form an ammonium salt, an alkali metal salt such as a sodium salt or a potassium salt, a salt such as an alkaline earth metal salt such as a calcium salt or a magnesium salt, a salt of organic bases such as dicyclohexylamine, N-methyl-D-glucamine, and tris(hydroxymethyl) methylamine, and an amino acid salt such as arginine and lysine, and more preferably, examples of the salt of stearic acid include magnesium stearate, lithium stearate, tin(II) stearate, and the like, but is not limited thereto.

The pharmaceutical composition may further include a pharmaceutically acceptable carrier. The pharmaceutically acceptable carrier is typically used in the formulation of a drug, and may be one or more selected from the group consisting of lactose, dextrose, sucrose, sorbitol, mannitol, starch, gum acacia, calcium phosphate, alginate, gelatin, calcium silicate, microcrystalline cellulose, polyvinylpyrrolidone, cellulose, water, syrup, methyl cellulose, methyl hydroxybenzoate, propyl hydroxybenzoate, talc, magnesium stearate, mineral oil, and the like, but is not limited thereto. The pharmaceutical composition may further include one or more selected from the group consisting of a diluent, an excipient, a lubricant, a wetting agent, a sweetener, a flavoring agent, an emulsifier, a suspending agent, a preservative and the like, which are typically used in the preparation of the pharmaceutical composition, in addition to the aforementioned ingredients.

The pharmaceutical composition, or an active ingredient stearic acid, or a salt of the stearic acid, or a prodrug of the stearic acid may be administered orally or parenterally. In the case of parenteral administration, the pharmaceutical composition or the active ingredient may be administered by intravenous injection, subcutaneous injection, intramuscular injection, peritoneal injection, endothelial administration, local administration, intranasal administration, intrapulmonary administration, rectal administration, or the like.

As used herein, the term “pharmaceutically effective amount” refers to an amount of an active ingredient capable of exerting a pharmaceutically meaningful effect. A pharmaceutically effective amount of the active ingredient for a single dose may be prescribed in various ways depending on factors, such as formulation method, administration method, age, body weight, sex or disease condition of the patient, diet, administration time, administration interval, administration route, excretion rate and response sensitivity. For example, a pharmaceutically effective amount of stearic acid for a single dose may range from 0.0001 to 200 mg/kg, 0.001 to 100 mg/kg, or 0.02 to 10 mg/kg, but is not limited thereto, previously licensed drugs pirfenidone and nintedanib or other publicly-known pulmonary fibrosis inhibitors may be used together in an effective amount previously licensed or known in the art, and it is obvious to those skilled in the art that the dose may be adjusted more or less than when administered alone, depending on the use examples and proportions disclosed in the present invention.

The pharmaceutical composition, or an active ingredient stearic acid, or a salt of the stearic acid, or a prodrug of the stearic acid, or a pulmonary fibrosis inhibitor may be formulated in the form of a solution, a suspension, a syrup or an emulsion in an oil or aqueous medium, or in the form of an extract, an acida, a powder, a granule, a tablet, a capsule, or the like, and may further include a dispersant or a stabilizer for formulation.

In addition, the present invention provides a method for preventing or treating pulmonary fibrosis, the method comprising: administering, to an individual, (i) stearic acid, a salt of the stearic acid or a prodrug of the stearic acid; and (ii) a pulmonary fibrosis inhibitor.

In the present invention, a plurality of ingredients such as stearic acid, a salt of the stearic acid or a prodrug of the stearic acid; and a pulmonary fibrosis inhibitor may be formulated together or individually, and may also be administered to an individual simultaneously, sequentially, or individually.

The individual subjected to prevention and/or treatment may be a mammal, for example, a primate including a human, a monkey, and the like, a rodent including a mouse, a rat, and the like, or a cell or tissue isolated from the living organism thereof. In an example, the subject is a mammal suffering from pulmonary fibrosis, for example, idiopathic pulmonary fibrosis, for example, a primate including a human, a monkey, and the like, a rodent including a mouse, a rat, and the like, or a cell or tissue isolated from the living organism thereof.

As still another aspect of the present invention, the present invention provides a food composition for preventing or treating pulmonary fibrosis, the composition comprising, as active ingredients: (i) stearic acid, a salt of the stearic acid or a prodrug of the stearic acid; and (ii) a pulmonary fibrosis inhibitor.

When the composition of the present invention is prepared as a food composition, the composition of the present invention may include ingredients typically added during the production of food, and may include, for example, protein, carbohydrate, fat, nutrient, seasoning, and a flavoring agent. Examples of the above-described carbohydrate include typical sugars such as monosaccharides, for example, glucose, fructose and the like; disaccharides, for example, maltose, sucrose and the like; and polysaccharides, for example, dextrin, cyclodextrin and the like, and sugar alcohols such as xylitol, sorbitol, and erythritol. As the flavoring agent, it is possible to use a natural flavoring agent (thaumatin, stevia extract [for example, rebaudioside A, glycyrrhizin and the like]) and/or a synthetic flavoring agent (saccharin, aspartame, and the like).

For example, when the food composition of the present invention is prepared as a drink, the composition may further include citric acid, liquid fructose, sugar, sucrose, acetic acid, malic acid, a fruit juice, a legume extract, a jujube extract, a licorice extract, and the like.

As used herein the term salt refers to a formation of an active ingredient which does not induce serious irritation in the organism to which the active ingredient is administered and does not impair the biological activity and physical properties of the active ingredient. The salt may be obtained by reacting the active ingredient of the present invention with an inorganic acid such as hydrochloric acid, bromic acid, sulfuric acid, nitric acid, and phosphoric acid, a sulfonic acid such as methanesulfonic acid, ethanesulfonic acid, and p-toluenesulfonic acid, and an organic carbonic acid such as tartaric acid, formic acid, citric acid, acetic acid, trichloroacetic acid, trifluoroacetic acid, capric acid, isobutanoic acid, malonic acid, succinic acid, phthalic acid, gluconic acid, benzoic acid, lactic acid, fumaric acid, maleic acid, and salicyclic acid. Further, the salt may also be obtained by reacting the active ingredient of the present invention with a base to form an ammonium salt, an alkali metal salt such as a sodium salt or a potassium salt, a salt such as an alkaline earth metal salt such as a calcium salt or a magnesium salt, a salt of organic bases such as dicyclohexylamine, N-methyl-D-glucamine, and tris(hydroxymethyl) methylamine, and an amino acid salt such as arginine and lysine, but is not limited thereto.

The food composition of the present invention may be used as human food, animal feed, a feed additive, or the like.

According to yet another aspect of the present invention, the present invention provides a therapeutic aid for pulmonary fibrosis having resistance to a pulmonary fibrosis inhibitor, the aid comprising, as an active ingredient, stearic acid, a salt of the stearic acid or a prodrug of the stearic acid.

Existing pulmonary fibrosis inhibitors (for example, pirfenidone or nintedanib) may not show the desired delay, amelioration, or therapeutic effect of fibrosis despite continuous administration. Further, as an exemplary embodiment of the present invention, a significant improvement in the fibrosis index may be insignificant despite the administration of the aforementioned pulmonary fibrosis inhibitor. As described above, when the stearic acid of the present invention or a salt thereof is used as a therapeutic aid, it shows a significant improving effect on fibrosis indices such as COL-1 and α-SMA, and thus, may show a desired ameliorating and/or treating effect on fibrosis.

According to yet another aspect of the present invention, the present invention provides a method for providing information on whether or not to co-administer stearic acid, a salt of the stearic acid or a prodrug of the stearic acid, the method comprising the following steps:

(a) confirming the expression level of collagen 1 (COL-1) and α-SMA, which are fibrosis markers, in pulmonary fibroblasts isolated from patients to whom a pulmonary fibrosis inhibitor is administered;

(b) confirming the expression level of collagen 1 and α-SMA by co-treating the pulmonary fibroblasts with the pulmonary fibrosis inhibitor and stearic acid, a salt of the stearic acid, or a prodrug of the stearic acid; and

(c) determining that in the case of combined treatment of the pulmonary fibrosis inhibitor with the stearic acid, the salt of the stearic acid or the prodrug of the stearic acid, when the expression level of collagen 1 and α-SMA is decreased, stearic acid or a salt thereof can be administered.

In the present invention, the patient is not limited, and is preferably a mammal, more preferably a mammal selected from the group consisting of a human, a rat, a monkey, a dog, a cat, a cow, a horse, a pig, a sheep, and a goat, and most preferably a human.

The pulmonary fibroblasts included in the method of the present invention are not limited as long as they are naturally or artificially isolated from the patient and include the patient's fibrosis marker-related genetic information.

According to yet another aspect of the present invention, the present invention provides a pharmaceutical composition for inhibiting side effects by a pulmonary fibrosis inhibitor, the composition comprising, as an active ingredient, stearic acid, a salt of the stearic acid or a prodrug of the stearic acid.

According to the present invention, the composition including the stearic acid, the salt of the stearic acid or the prodrug of the stearic acid of the present invention may exhibit an effect of inhibiting side effects exhibited by existing pulmonary fibrosis inhibitors, for example, a reduction in body weight.

Hereinafter, preferred examples for helping the understanding of the present invention will be suggested. However, the following examples are provided only to more easily understand the present invention, and the contents of the present invention are not limited by the following examples.

### EXAMPLES

### Example 1. Experimental Preparation and Experimental Methods

<1-1> Preparation of Lung Tissue Sample

50 mg or less (about 50 mg) of each of lung tissues of patients with human idiopathic pulmonary fibrosis (n=10) and normal persons (n=10) (lung tissues purchased from the Bio-Resource Center of Asan Medical Center, Seoul, or collected by clinical researchers according to the Institutional Review Board (IRB) procedure) was homogenized using TissueLyzer (Qiagen), a small amount of hydrochloric acid was added thereto such that the concentration was 25 mM, and then a sample was extracted using iso-octane. Further, 50 μl of a 0.1 mg/mL internal standard fatty acid (Internal standard; heneicosanoic acid (C21:0) for free fatty acids) was added to the sample before extraction of free fatty acids, and the obtained sample was vacuum-centrifuged and dried after extraction of lipids. Next, the free fatty acids were derivatized for gas chromatography mass spectrometry (GC/MS) analysis. After the free fatty acids were reacted with BCl₃—MeOH at 60° C. for 30 minutes, the free fatty acids were methyl-esterified.

1-2. GC/MS Analysis

Fatty acid methyl esters were analyzed using an Agilent 7890/5975 GCMSD system (Agilent Technology) and HP-5MS 30 m×250 um (micrometer)×a 0.25 um column (Agilent 19091S-433), and He (99.999%) was used as a carrier gas. The initial temperature was set to 50° C., and after a hold time of 2 minutes, the temperature was increased to 120° C. at a rate of 10° C./min. Thereafter, the temperature was raised to 250° C. at a rate of 10° C./min and maintained for 15 minutes. Finally, the GC column was cleaned at 300° C., and a 5-minute solvent delay and a scan mode were applied. Thereafter, quantification was performed using an extracted ion chromatogram corresponding to a specific fatty acid, the ratio of the peak region of each fatty acid methyl ester/heneicosane methyl ester was determined, and a relative comparison between fatty acids was performed.

1-3. Pre-Treatment with Stearic Acid

After epithelial cells and fibroblasts were aliquoted into 6-well plates at 2×10⁴ cells/well and a stabilization time was imparted for 24 hours, and 15 hours after deficiency, cells were treated with stearic acid (40 uM/mL), TGF-β (5 ng/mL), and stearic acid (40 uM/mL)+TGF-β (5 ng/mL) in this order. After the cells were cultured in an incubator for 24 hours after the treatment, the next-step experiment was performed.

1-4. Cell Viability Analysis

After a 24-hour cell stimulation by the method in Example 1-3 was completed, the medium of epithelial cells and fibroblasts was replaced with a general culture medium, 10 μl of an MTT solution (20 mg/ml) was further added, and then the outside of the plate was wrapped with aluminum foil and cells were cultured in an incubator for 2 hours. After 2 hours, all the media inside the cells were removed, 100 μl of dimethyl sulfoxide (DMSO) was added thereto, and then the cells were cultured in an incubator for an additional 1 hour to disrupt the cells. After 2 hours, cell activation was measured at an absorbance value of 595 nm using an ELISA reader.

1-5. Measurement of Collagen 1 and α-SMA

After 24 hours of cell stimulation by the method of Example 1-3, the cells were washed twice with iced phosphate buffer saline (PBS), a protein lysate solution was put thereinto, the cells were scraped out and collected in a 1.5 ml EP tube, and then the cells were lysed in a grinder for 30 seconds. Next, centrifugation was performed at a speed of 14,000 rpm at 4° C. for 20 minutes, and the protein was quantified using the BCA analysis method. Thereafter, the protein sample was boiled at 95° C. for 10 minutes for the same amount of protein, and then the expression levels of collagen type 1 and α-SMA were measured by immunoblotting. After the expression level of the protein was confirmed, a significance test between samples was performed using a statistical program.

### Example 2. Therapeutic Effect of Stearic Acid on Idiopathic Pulmonary Fibrosis

### Example 2-1. Selection of Diagnostic Markers for Idiopathic Pulmonary Fibrosis

To select diagnostic markers for patients with idiopathic pulmonary fibrosis (IPF), free fatty acids in lung tissues from a normal group (Normal) and from a group of patients with idiopathic pulmonary fibrosis (IPF) were quantified, and the average value of the measured free fatty acid contents in the lung tissue is illustrated in FIG. 1.

As illustrated in FIG. 1, it was confirmed that in the case of palmitoleic acid (C16:1), palmitic acid (C16:0), linoleic acid (C18:2), and oleic acid (C18:1), the content in the lung tissue of the group of patients with idiopathic pulmonary fibrosis was remarkably increased compared to the normal group, whereas in the case of stearic acid (C18:0), the content in the lung tissue of the group of patients with idiopathic pulmonary fibrosis was significantly decreased compared to the normal group (p=0.017). Meanwhile, in the case of myristic acid (C14:0), which is a saturated fatty acid having 14 carbon atoms, arachidonic acid (C20:4), which is an unsaturated fatty acid having 20 carbon atoms, eicosapentaenoic acid (EPA; C20:5), and docosahexaenoic acid (DHA; C22:6), there was no clear difference in content in lung tissue between the group of patients with idiopathic pulmonary fibrosis and the normal group. Based on these results, the present inventors selected stearic acid as a diagnostic marker for patients with idiopathic pulmonary fibrosis.

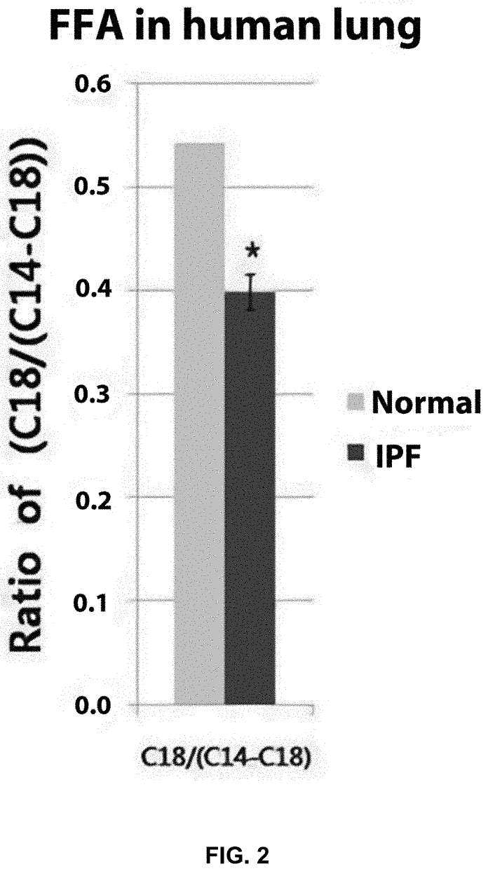

In addition, as can be seen in FIG. 1, it was found that the total amount of saturated and unsaturated glass fatty acids having 18 or less carbon atoms except for stearic acid obtained by quantifying the free fatty acids in the lung tissue of the group of patients with idiopathic pulmonary fibrosis was increased compared to that in the lung tissue of the normal group. Thus, a value (content of stearic acid/total amount of C14-C18) obtained by dividing the content of stearic acid (C18:0) in the lung tissue by the sum of the saturated and unsaturated free fatty acids having 14 to 18 carbons (myristic acid (C14:0), palmitoleic acid (C16:1), palmitic acid (C16:0), linolenic acid (C18:2), oleic acid (C18:1) and stearic acid (C18:0)) is illustrated in FIG. 2.

As illustrated in FIG. 2, it was confirmed that the ratio of (content of stearic acid/total amount of C14-C18) in the lung tissue of the group of patients with idiopathic pulmonary fibrosis was significantly reduced compared to the normal group lung tissue (p=0.007). Therefore, these results suggest that the ratio of (content of stearic acid/total amount of C14-C18) in the lung tissue as well as the content of stearic acid in the lung tissue can be proposed as an index for diagnosis in patients with idiopathic pulmonary fibrosis.

### Example 2-2. Therapeutic Effect of Stearic Acid on Idiopathic Pulmonary Fibrosis

As confirmed in the results of Example 2-1, focusing on the reduction in content of stearic acid in the lung tissues of the patients with idiopathic pulmonary fibrosis, the present inventors tried to investigate the efficacy of stearic acid as a therapeutic agent as well as a diagnostic marker for idiopathic pulmonary fibrosis by verifying whether the therapeutic effect appears during administration of stearic acid to patients with idiopathic pulmonary fibrosis.

The characteristics of pulmonary cells in patients with idiopathic pulmonary fibrosis are known to be activation of fibroblasts and loss of epithelial cells by transforming growth factor (TGF)-β. Based on these facts, the effects by treatment with stearic acid were tested by treating pulmonary fibroblasts and pulmonary epithelial cells with TGF-β to create an environment similar to idiopathic pulmonary fibrosis.

For this purpose, after each culture (BEGM(Lonza) in the case of MRC-5, and BMEM (ATCC) in the case of BEAS-2B) of human pulmonary fibroblasts (MRC-5; ATCC® CCL171™) and human pulmonary epithelial cells (BEAS-2B; ATCC® CRL9609™) was treated with stearic acid (40 uM/mL), TGF-β (5 ng/mL; Sigma), or stearic acid (40 uM/mL)+TGF-β (5 ng/mL) by the method described in Example 1-3 for 24 hours, cell viability was measured by the method in Example 1-4. In this case, as a negative control for comparison, the cell viability in (medium only) cell culture untreated with both stearic acid and TGF-β was measured by the same method as described above. The results obtained above are illustrated in FIG. 3 (CTL: control (medium only), SA: stearic acid 40 uM/mL treatment group, TGF-b: TGF-β 5 ng/mL treatment group, SA+TGF-b; stearic acid 40 uM/mL and TGF-β 5 ng/mL treatment group), FIG. 3A illustrates the cell viability (%) of pulmonary fibroblasts, and FIG. 3B illustrates the cell viability (%) of pulmonary epithelial cells. Further, in the above results, the cell viability in each test group was shown as a relative value to a cell viability of 100% in the control (CTL).

As a result, as illustrated in FIG. 3A, in the case of pulmonary fibroblasts, cell viability increased when the pulmonary fibroblasts were treated with TGF-β alone, and cell viability decreased when the pulmonary fibroblasts were co-treated with stearic acid and TGF-β. In contrast, as illustrated in FIG. 3B, in the case of pulmonary epithelial cells, cell viability decreased when the pulmonary epithelial cells were treated with TGF-β alone, and cell viability increased when the pulmonary epithelial cells were co-treated with stearic acid and TGF-β. These results show that stearic acid can inhibit the activation of pulmonary fibroblasts and the loss of pulmonary epithelial cells by TGF-β, showing the therapeutic effect of stearic acid on idiopathic pulmonary fibrosis, which can be characterized by the activation of pulmonary fibroblasts and the loss of pulmonary epithelial cells by TGF-β.

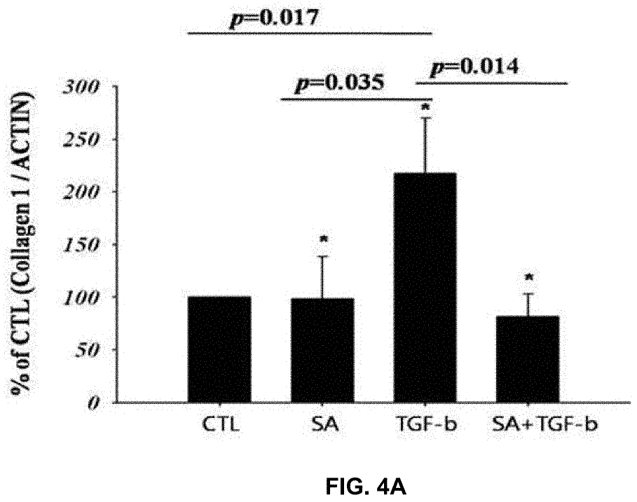

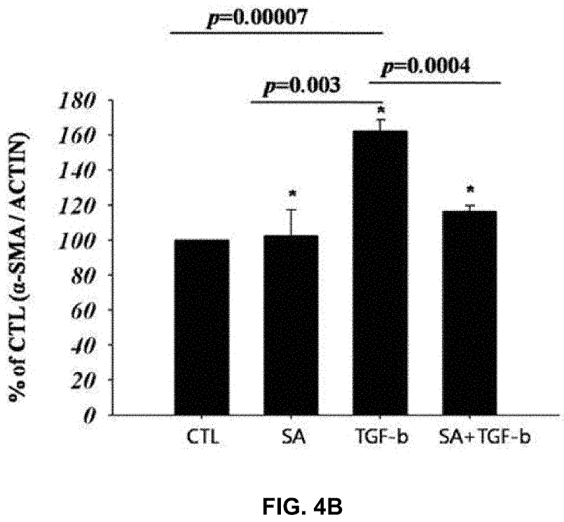

Further, changes in collagen 1 (FIG. 4A) and alpha-smooth muscle actin (α-SMA) (FIG. 4B), which are markers of fibrosis, caused by stearic acid, were observed in pulmonary fibroblasts. The collagen 1/actin or α-SMA/actin indicated on the y-axis of FIGS. 4A and 4B means a value obtained by correcting the protein amount of collagen 1 or α-SMA with the amount of actin, which is an intracellular control protein. As a result, as illustrated in each of FIGS. 4A and 4B, it was confirmed that when compared to the pulmonary fibroblast control (CTL) that was not treated with stearic acid or TGF-β, collagen 1 and α-SMA were significantly increased when treated with only TGF-β, which is known as a mechanistic material of pulmonary fibrosis, whereas this change was inhibited by treatment with stearic acid. The results show the pulmonary fibrosis inhibitory effect of stearic acid.

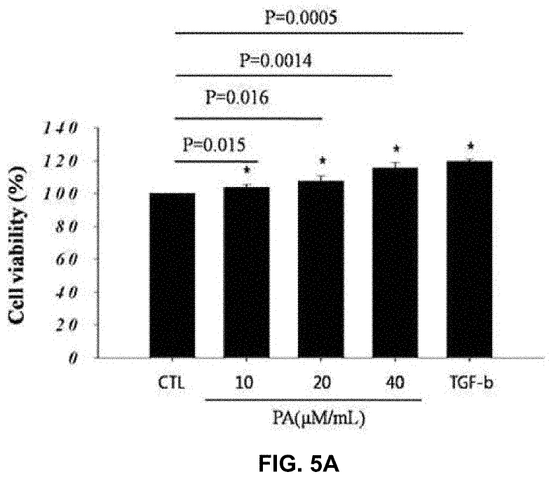

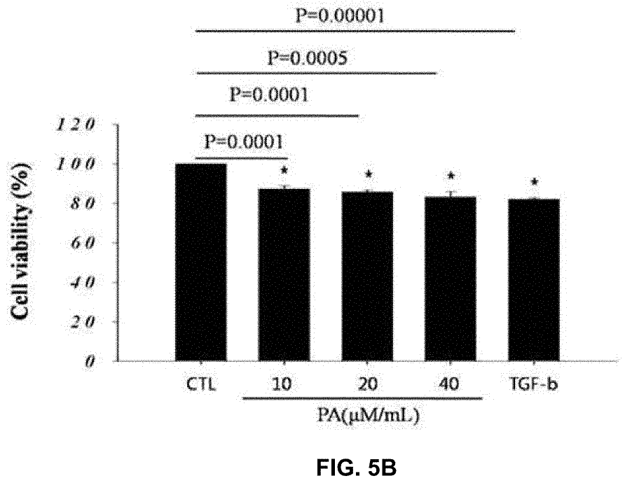

Furthermore, since it was observed that stearic acid was decreased in pulmonary tissues of patients with idiopathic pulmonary fibrosis, whereas other saturated and unsaturated fatty acids including C14 to C18 carbon atoms, such as palmitic acid, were increased in Example 2-1, cell viability was measured after treatment with palm itic acid (PA) observed to be increased in patients with pulmonary fibrosis at various concentrations (10, 20, and 40 μM/mL) in order to verify the therapeutic effect of stearic acid in the treatment of pulmonary fibrosis. As a result, as can be seen in FIG. 5A, when pulmonary fibroblasts were treated with palmitic acid, cell viability was increased according to the concentration of palm itic acid, and it was shown through FIG. 5B that the cell viability of pulmonary epithelial cells was decreased according to the treatment concentration of palmitic acid. These results show that during treatment with palmitic acid at high concentration, the same levels of results as those for TGF-β are induced.

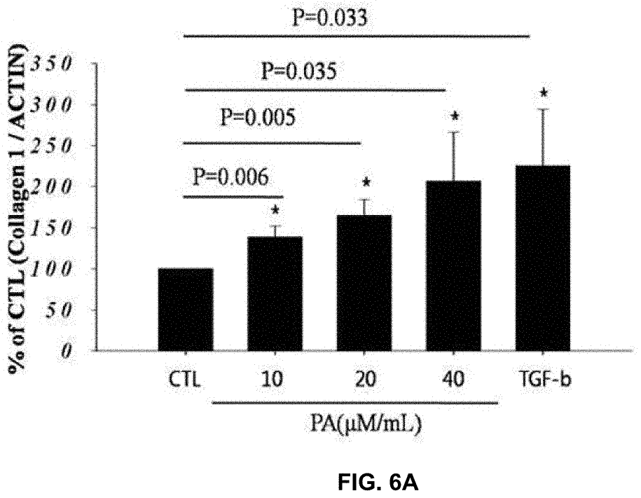

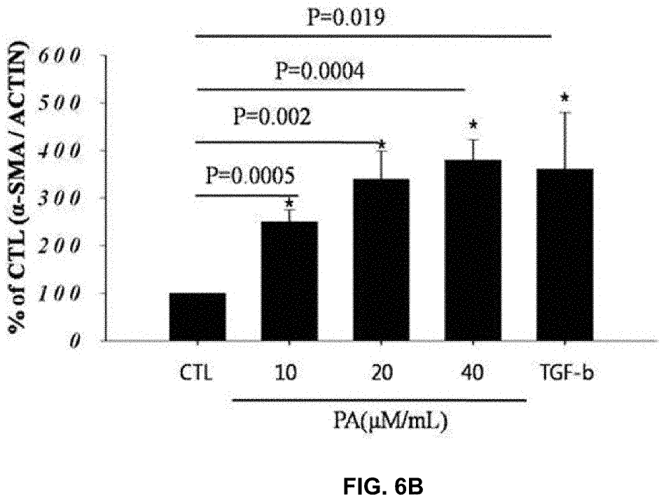

Further, referring to the test method of obtaining the results in FIG. 4, after pulmonary fibroblasts were treated with palmitic acid at various concentrations (10, 20, and 40 μM/mL), the levels of collagen 1 (collagen 1/actin; FIG. 6A) and α-SMA (α-SMA/actin; FIG. 6B), which are intracellular fibrosis markers, were measured, and shown as relative values to the control (CTL; medium only). As a result, as illustrated in FIGS. 6A and 6B, it was confirmed that when pulmonary fibroblasts were treated with palmitic acid, both collagen 1 and α-SMA were increased at levels similar to that in the case where pulmonary fibroblasts were treated with only TGF-β, which is known to be a mechanistic material of idiopathic pulmonary fibrosis, unlike during the treatment with stearic acid alone in FIGS. 4A and 4B.

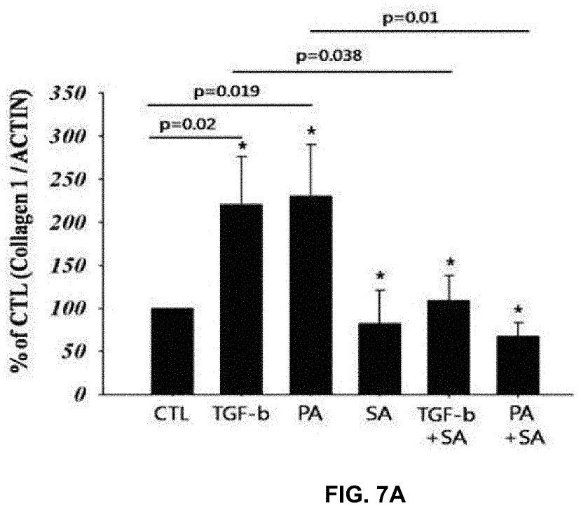

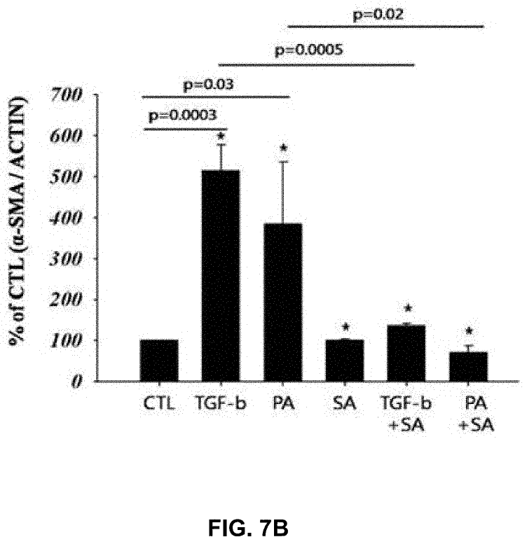

The present inventors measured the levels of collagen 1 ((collagen 1/actin); FIG. 7A) and α-SMA (α-SMA/actin; FIG. 7B), and showed the levels as relative values to the control (CTL; medium only) in order to verify the inhibitory effects of stearic acid (SA) on the pulmonary fibrosis caused by palmitic acid (PA) shown to activate pulmonary fibrosis in FIGS. 5 and 6. 40 uM/mL stearic acid, 10 uM/mL palmitic acid, and 5 ng/mL TGF-β were used in the experiment, respectively. As a result of the experiment, as illustrated in FIGS. 7A and 7B, it was confirmed that the fibrosis increased by palmitic acid and TGF-β respectively was significantly inhibited by the combined treatment with stearic acid.

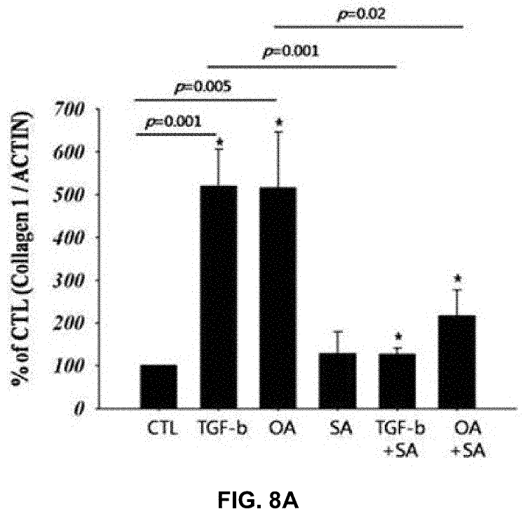

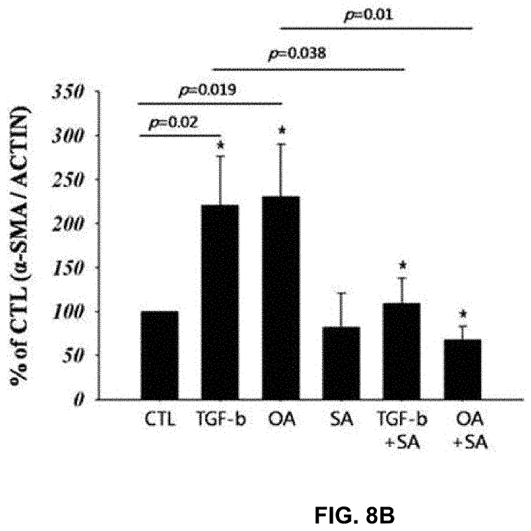

In addition, referring to the test method of obtaining the results in FIG. 7, the experiment was performed using oleic acid (OA) instead of palmitic acid, and the levels of collagen 1 (collagen 1/actin; FIG. 8A) and α-SMA (α-SMA/actin; FIG. 8B) were measured and shown as relative values to the control (CTL; medium only). 40 uM/mL stearic acid, 40 uM/mL oleic acid, and 5 ng/mL TGF-β were used in the experiment, respectively. As a result of the experiment, as illustrated in FIGS. 8A and 8B, it can be seen that similar to the results of palmitic acid, oleic acid also activated pulmonary fibrosis at the same level as in TGF-β, and as described above, it was confirmed that the pulmonary fibrosis increased by the treatment with TGF-β and oleic acid respectively was significantly inhibited by the treatment with stearic acid.

### Example 2-3. Anti-Fibrotic Effect of Stearic Acid in Bleomycin-Induced Pulmonary Fibrosis Animal Models

Based on the results of Example 2-2, the present inventors attempted to verify the anti-fibrotic effect of stearic acid in an animal model in which pulmonary fibrosis was induced by bleomycin. For this purpose, 6-week-old mice (C57BL6J) were classified into the following 4 groups of 4 or 5 mice, respectively: groups treated with (1) intratracheal saline+vehicle, (2) intratracheal saline+stearic acid, (3) intratracheal 4 units/kg bleomycin+vehicle, and (4) intratracheal bleomycin+stearic acid. Subsequently, mice were anesthetized with 50 mg/kg Alfaxan and 5 mg/kg Rompun, followed by infusion of bleomycin and saline into the trachea. The mice were treated with 3 mg/kg stearic acid using oral gavage (zonde) three times a week for 3 weeks. Thereafter, on day 21, lung tissues and blood were collected from the mice and used for the study.

As a result of the experiment, as illustrated in 9A, it was confirmed that stearic acid exhibited an effect of inhibiting a reduction in body weight due to bleomycin. More specifically, a sharp reduction in body weight was observed in the bleomycin treatment group (Bleo) on day 7, and then a pattern of an increase in body weight was observed, but a significant reduction in body weight was continuously observed compared to the control. In contrast, it was confirmed that when stearic acid (SA) was administered together, the sharp reduction in body weight due to bleomycin on day 7 was significantly inhibited.

In addition, as a result of observing whether stearic acid alleviates the histopathological characteristics due to bleomycin-induced fibrosis, as illustrated in 9B, characteristics of the normal lung tissue were well observed in the control (Saline), but it was observed that histopathological characteristics of pulmonary fibrosis such as cell compactness, alveolar wall thickening, and alveolar space remodeling appeared in the bleomycin treatment group (Bleomycin). In contrast, it was confirmed that the histopathological characteristics of pulmonary fibrosis were remarkably reduced in the group treated with both bleomycin and stearic acid.

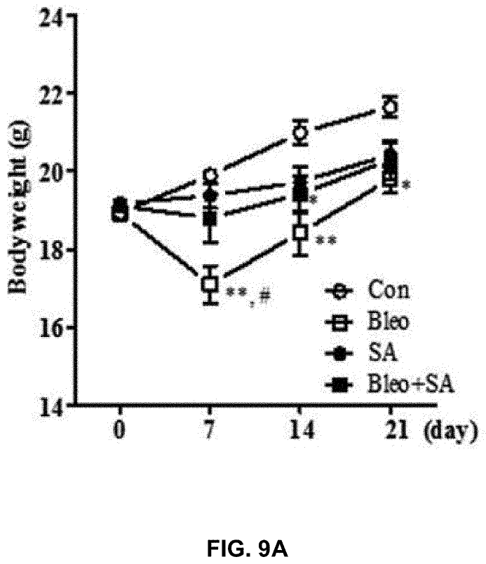

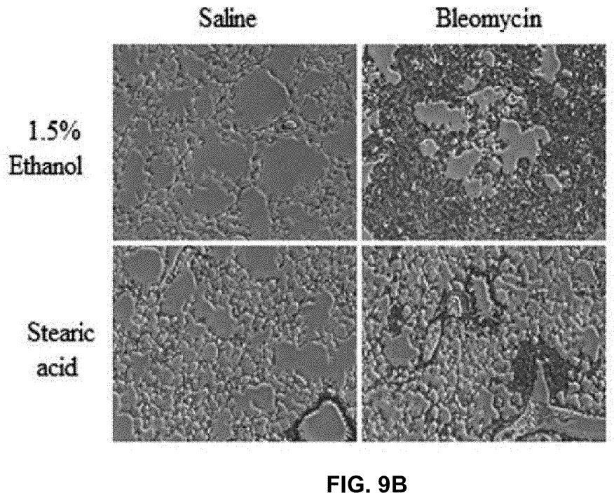

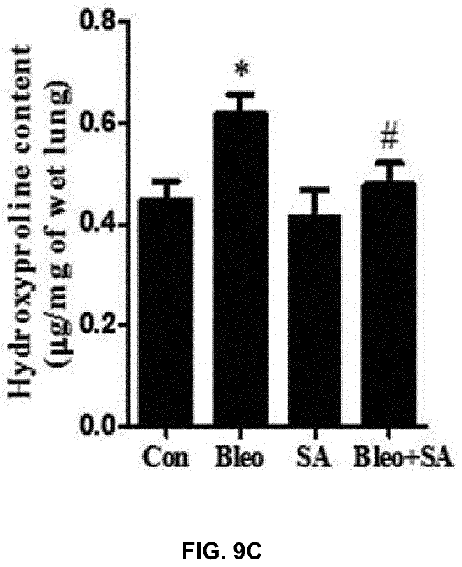

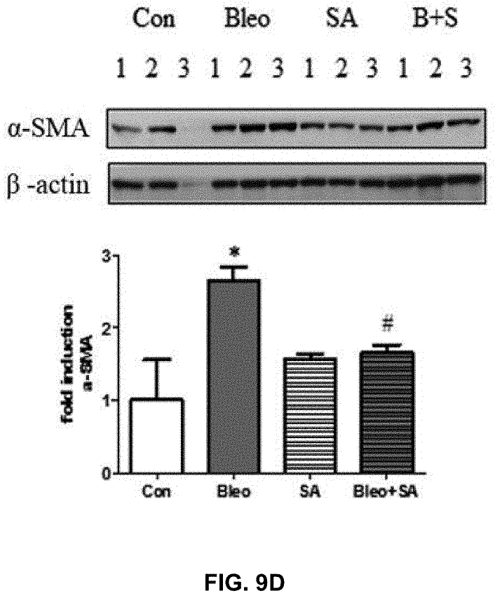

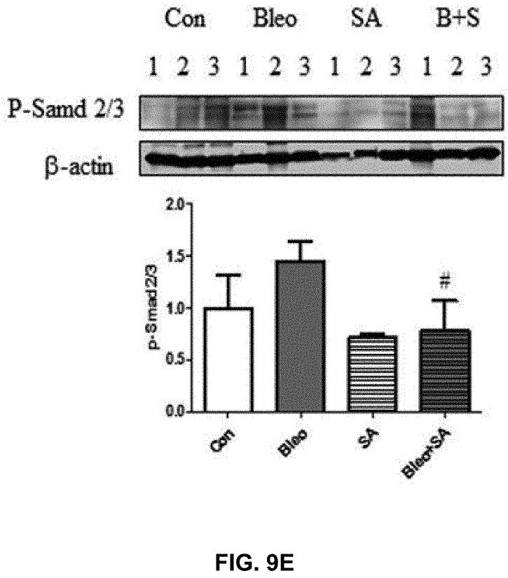

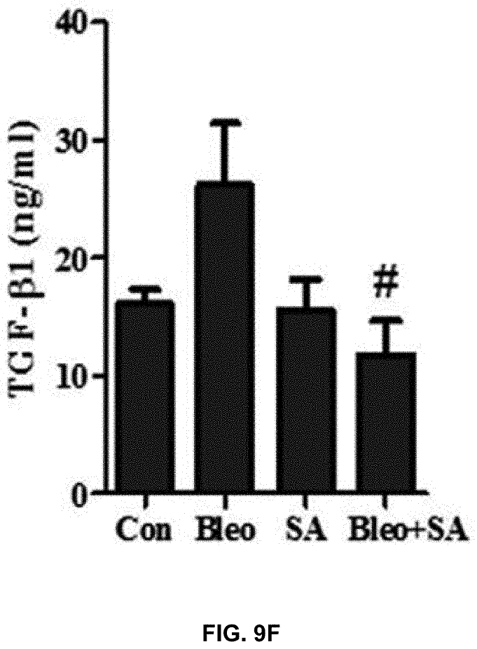

In addition, as can be seen in FIGS. 9C to 9F, it was confirmed that stearic acid exhibited the effects of inhibiting the accumulation of hydroxyproline, which is a major component in collagen in tissue, due to bleomycin (FIG. 9C), inhibiting an increase in expression of α-SMA due to bleomycin in lung tissues (FIG. 9D), inhibiting Smad signaling due to bleomycin (inhibition of an increase in expression of p-Smad2/3)(FIG. 9E), and inhibiting an increase in the blood level of TGF-β1 induced by bleomycin (FIG. 9F).

The results suggest that stearic acid shows an anti-fibrotic effect by inhibiting the expression of p-Smad2/3 increased by TGF-β.

### Example 2-4. Anti-Fibrotic Effect of Stearic Acid in Human Primary Fibroblasts

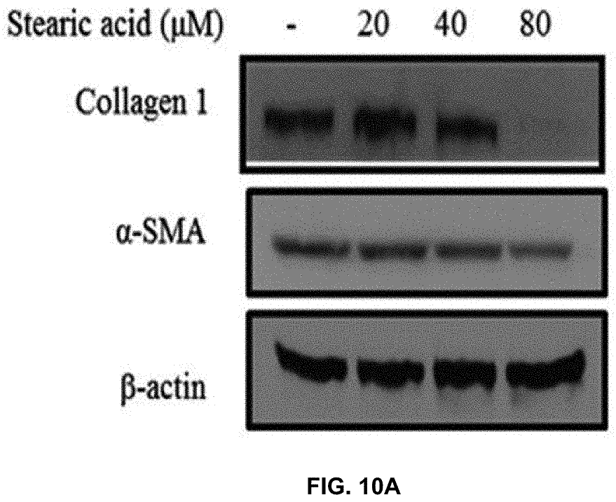

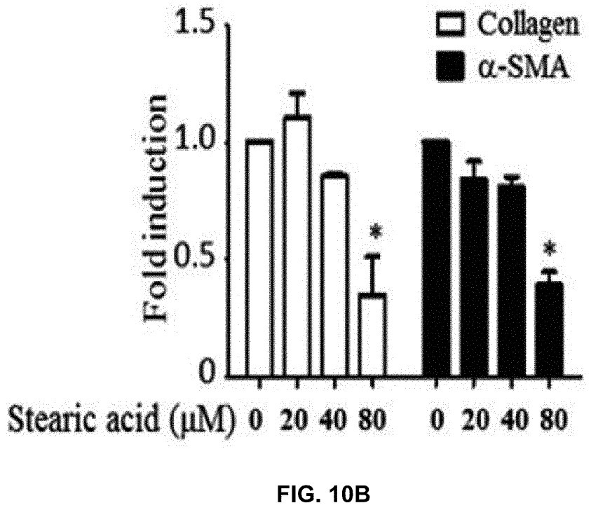

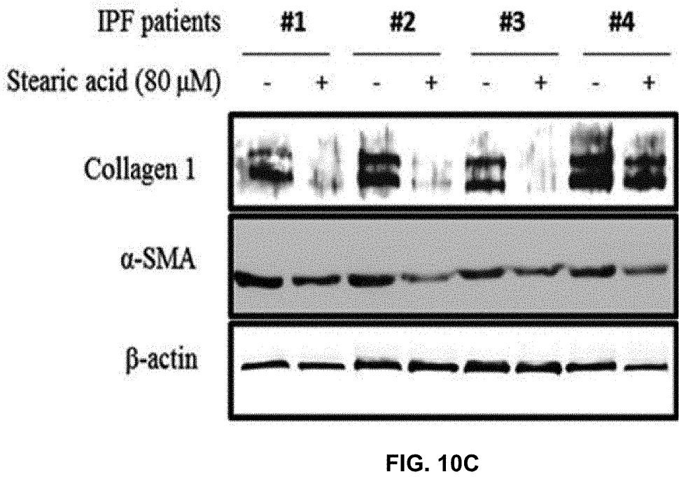

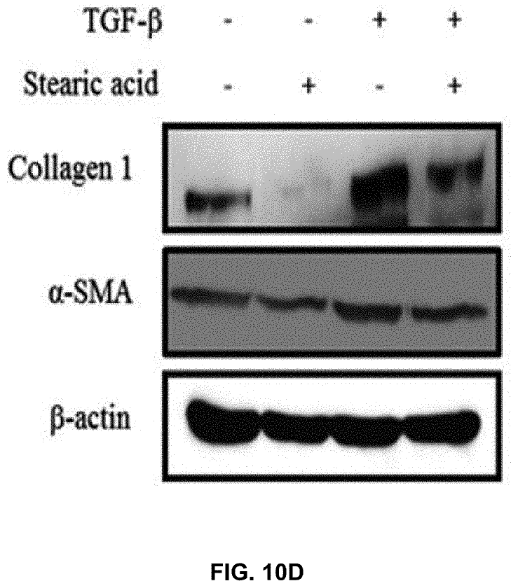

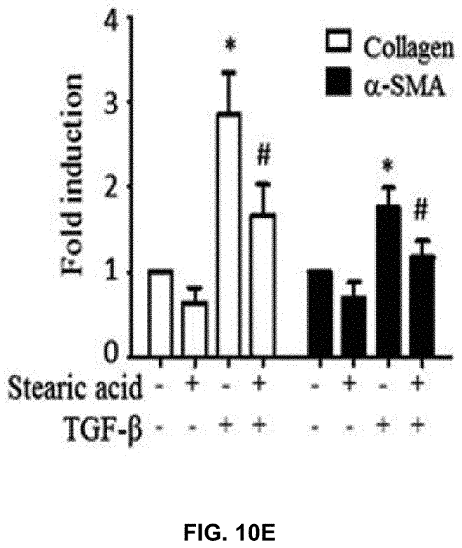

In addition to the results in the Examples, the present inventors sought to verify the anti-fibrotic effect of stearic acid on fibroblasts derived from lung tissues in patients with idiopathic pulmonary fibrosis (IPF). For this purpose, after primary fibroblasts were isolated from lung tissues of the patients, and then the cells were treated with stearic acid at various concentrations for 24 hours, the expression levels of collagen 1 and α-SMA were measured (FIGS. 10A and 10B), fibroblasts obtained from 4 patients were treated with 80 μM stearic acid for 24 hours, and then the expression levels of collagen 1 and α-SMA were measured (FIG. 10C). In addition, after the expression of collagen type 1 and α-SMA was increased by inducing the fibrosis caused by TGF-β1 in patient-derived fibroblasts, the anti-fibrotic effect of stearic acid was verified (FIGS. 10D and 10E).

As a result of the experiment, as illustrated in FIGS. 10A and 10B, it was confirmed that the basal level expression of collagen1 and α-SMA was significantly reduced in the human-derived primary fibroblasts when treated with 80 μM stearic acid, and as can be seen in FIG. 10C, it was shown that when primary fibroblasts obtained from 4 patients with IPF were treated with 80 μM stearic acid, the basal level expression of both collagen 1 and α-SMA was significantly reduced, and as illustrated in FIGS. 10D and 10E, it was confirmed that even when the fibrosis by TGF-β1 was induced in patient-derived fibroblasts, the expression of collagen 1 and α-SMA was significantly reduced by the treatment with 80 μM stearic acid.

### Example 2-5. Confirmation of the Role of Stearic Acid in Epithelial Cells

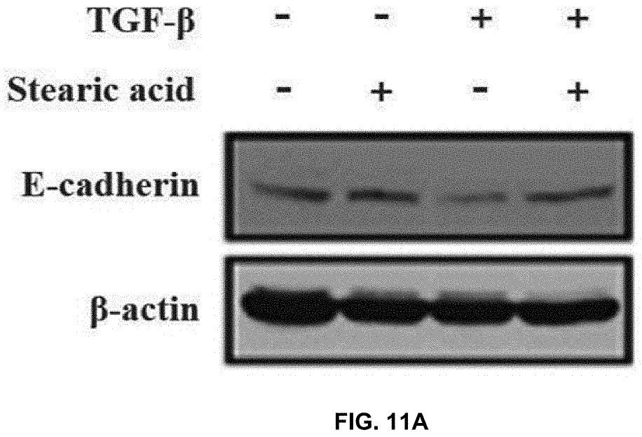

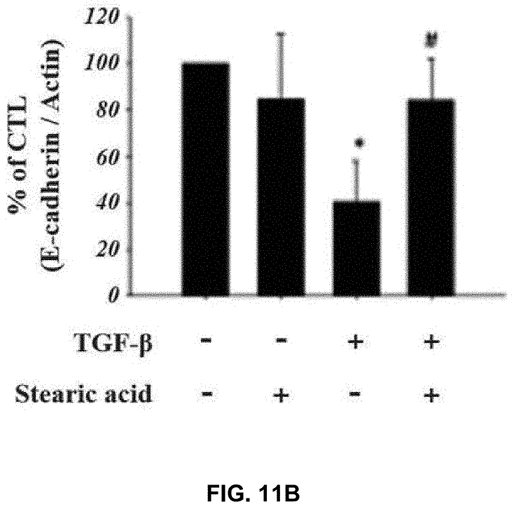

The present inventors examined the expression level of E-cadherin after treating Beas-2B, which is a human pulmonary epithelial cell line, with TGF-β1 and/or 40 μM stearic acid for 24 hours in order to examine the effects of stearic acid on epithelial cells. As a result, as illustrated in FIGS. 11A and 11B, it was confirmed that when Beas-2B was treated with 40 μM stearic acid, the expression of E-cadherin reduced by TGF-β1 was restored in Beas-2B cells. It is known that when epithelial cells are treated with TGF-β1, the number of epithelial cells is decreased while epithelial cells are differentiated into fibroblasts due to EMT, and when EMT occurs, the expression level of E-cadherin serving to maintain the function of epithelial cells is also decreased. Thus, through the results, it can be seen that when epithelial cells are treated with stearic acid, the increase in EMT due to the treatment with TGF-β1 is inhibited, and the expression level of E-cadherin is significantly increased. It was confirmed in FIG. 3B that when epithelial cells are treated with TGF-β1, the proliferation of epithelial cells was inhibited and the proliferation of epithelial cells was restored by stearic acid.

### Example 2-6. Elucidation of Anti-Fibrotic Mechanism of Stearic Acid in Fibroblasts

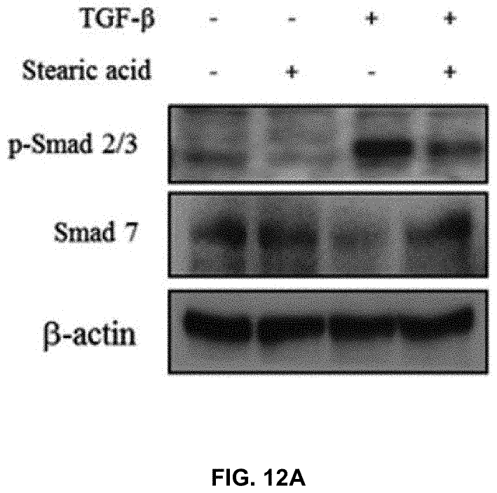

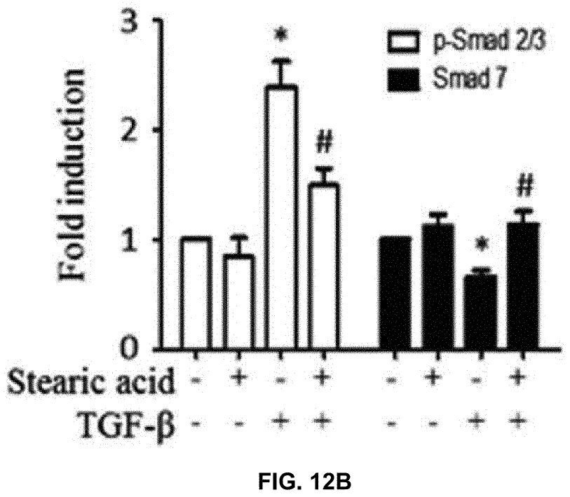

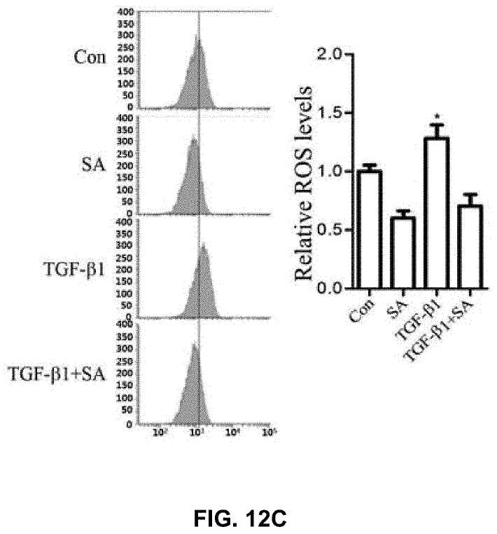

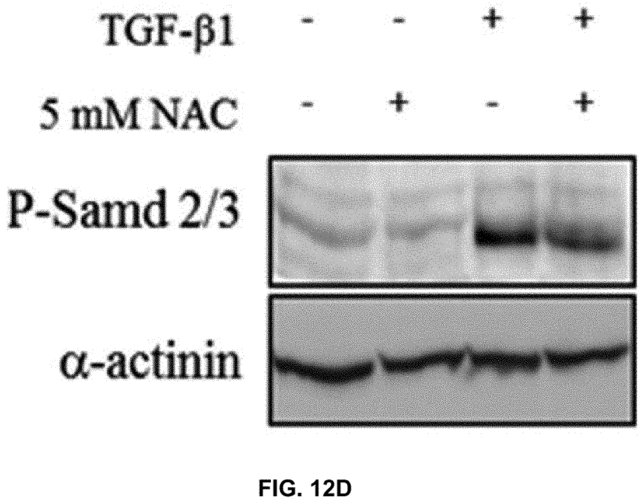

The present inventors pre-treated a human pulmonary fibroblast cell line MRC-5 with 40 μM stearic acid for 16 hours, treated the MRC-5 cells with TGF-β1 for 1 hour, and then examined the expression levels of p-Smad2/3 and Smad7 in order to elucidate the anti-fibrotic mechanism of stearic acid in human pulmonary fibroblasts (FIGS. 12A and 12B). Further, to investigate the effect of stearic acid on the production of reactive oxygen species (ROS), MRC-5 cells were pre-treated with 40 μM stearic acid for 16 hours, and cells treated with TGF-β1 for 1 hour were stained with DCF-DA and analyzed by FACS (FIG. 12C). Furthermore, MRC-5 cells were pre-treated with 5 mM N-acetylcysteine (NAC), which is an antioxidant, for 1 hour and treated with TGF-β1 for 1 hour, and then the expression of p-Smad2/3 was examined (FIG. 12D).

As a result of the experiments, as illustrated in FIGS. 12A and 12B, it was confirmed that stearic acid inhibited the expression of p-Smad2/3 induced by TGF-β1 in MRC-5 cells and restored the expression of Smad 7 reduced by TGF-β1, and as can be seen in FIG. 12C, it was confirmed that stearic acid remarkably reduced the level of reactive oxygen species induced by TGF-β1 in MRC-5 cells. Furthermore, as illustrated in FIG. 12D, it was confirmed that an antioxidant NAC inhibited the expression of p-Smad2/3 induced by TGF-β1 in MRC-5 cells.

Through these results, it can be seen that stearic acid suppressed the production of ROS by inhibiting the expression of p-Smad2/3 induced by TGF-β1.

### Example 3: Therapeutic Effect on Idiopathic Pulmonary Fibrosis by Combined Administration of Stearic Acid and Existing Pulmonary Fibrosis Inhibitor Drug

The present inventors confirmed through Example 2 that stearic acid exhibited the anti-fibrotic effect, and thus, furthermore, the present inventors tried to see whether a synergistic therapeutic effect could be exhibited on idiopathic pulmonary fibrosis when stearic acid was co-administered with a drug used as an existing therapeutic agent for pulmonary fibrosis.

The primary fibroblasts derived from the patients with idiopathic pulmonary fibrosis used in the following experiment were cultured for 7 to 10 days while cutting the lung tissue of the patient into 1×1 mm² slices, and then periodically exchanging a cell culture solution (Eagle's minimal essential medium; EMEM) supplemented with 100 unit/ml penicillin, 100 μg/ml streptomycin, and 10% fetal bovine serum (FBS) under conditions of 5% CO₂ and 37° C., and cells of passage 2 to 5 were used for the experiment.

3-1. Verification of Anti-Fibrotic Effect by Combined Treatment of Stearic Acid and Pirfenidone

3-1-1. Anti-Fibrotic Effect by Combined Treatment in Human Pulmonary Fibroblasts

To verify the anti-fibrotic effect by the combined treatment of stearic acid and pirfenidone, which is a therapeutic agent for idiopathic pulmonary fibrosis, the human primary fibroblasts isolated by the above-described method were respectively or simultaneously treated with 5 ng/ml TGF-β, 40 μM stearic acid, and 400 or 800 μM pirfenidone for 24 hours, and then the expression levels of collagen type 1 (COL-1) and α-SMA, which are markers of fibrosis, were measured by western blotting, and the inhibitory rate was quantitatively analyzed by correcting the amount of each protein with the amount of actin, which is an intracellular control protein.

As a result, as illustrated in FIG. 13, when compared to the case where cells were treated with TGF-β alone (Lane 3), it was observed that the reduction in COL-1 and α-SMA proteins was clearly exhibited in the case where cells were co-treated with stearic acid and pirfenidone (Lane 8), compared to a 400 μM pirfenidone single treatment group (Lane 7). It was confirmed that even when cells were treated with 800 μM pirfenidone, COL-1 and α-SMA proteins were decreased in the same manner as above when cells were co-treated with stearic acid and pirfenidone (Lane 12) compared to when cells were treated with pirfenidone alone (Lane 11). In contrast, in the case of the stearic acid single treatment group (Lane 4), α-SMA was reduced, but the change in COL-1 which is another marker of fibrosis, was insignificant. Further, as a result of quantitative analysis, it was confirmed that when the TGF-β single treatment group (TGF) was set to 100%, the inhibitory rate of COL-1 was increased to 157% in the pirfenidone single treatment group (TGF+PIR), but the inhibitory rate of COL-1 was remarkably increased to 187% in the combined treatment group with stearic acid (TGF+Combi).

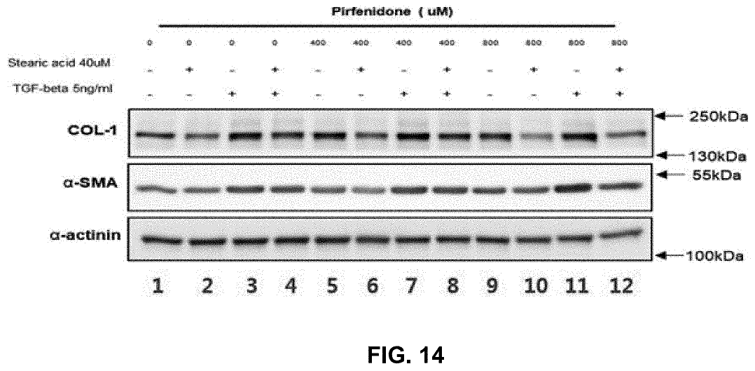

In addition, as a result of performing an experiment in the same manner as for MRC-5 which is a human pulmonary fibroblast cell line, as can be seen in FIG. 14, it was confirmed that when cells were treated with 800 μM pirfenidone, the reduction in COL-1 and α-SMA was clearly shown in the group co-treated with stearic acid and pirfenidone (Lane 12) compared to the single pirfenidone treatment group (Lane 11).

3-1-2. Anti-Fibrotic Effect by Combined Treatment in Human Pulmonary Epithelial Cells

In addition to the results of Example 3-1-1, the present inventors tried to analyze the degree of epithelial to mesenchymal transition (EMT), which is one of the indices for pulmonary fibrosis, during the combined treatment with stearic acid and pirfenidone by treating a human pulmonary epithelial cell line Beas-2B with 800 μM, and for this purpose, the expression level of fibronectin, which is one of the EMT markers, was measured.

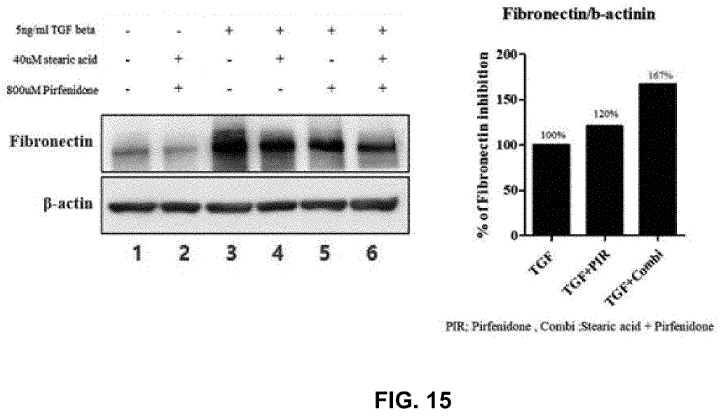

As a result, as illustrated in FIG. 15, a significant reduction in fibronectin was observed in the group co-treated with stearic acid and pirfenidone (Lane 6) compared to the group treated with pirfenidone alone (Lane 5). Furthermore, through the quantitative analysis results, the expression of fibronectin was decreased to about 120% in the pirfenidone single treatment group (TGF+PIR), whereas the expression of fibronectin was inhibited to 167% in the combined treatment group (TGF+Combi) confirming an excellent inhibitory effect.

3-1-3. Anti-Fibrotic Effect by Combined Treatment in Pulmonary Fibrosis Animal Model

In addition to the results of the above examples, the present inventors sought to confirm the anti-fibrotic effect by the combined treatment of stearic acid and pirfenidone in a pulmonary fibrosis animal model. For this purpose, 8-week-old mice (C57BL/6J) were anesthetized with 50 mg/kg Alfaxan and 5 mg/kg Rompun, followed by injection of bleomycin and saline into the trachea. From 7 days after administration of bleomycin, 3 mg/kg stearic acid, 300 mg/kg pirfenidone or the two drugs were orally administered at the same time once every 2 days for 2 weeks, and changes in mouse body weight were measured up to 21 days after administration of bleomycin.

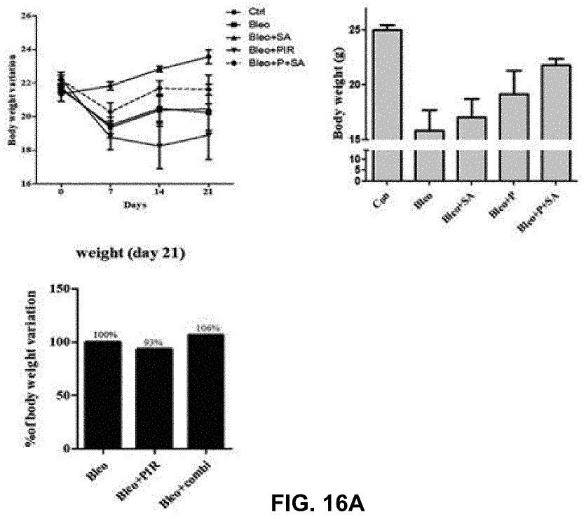

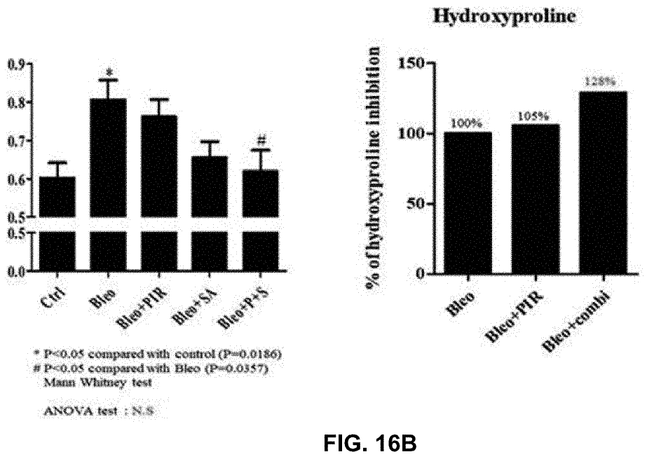

As a result of the experiment, as illustrated in FIG. 16A, it was confirmed that in the group in which pulmonary fibrosis was induced by administration of bleomycin (Bleo), the body weight was reduced compared to the normal control (Ctrl), and in the group treated with pirfenidone alone (Bleo+PIR), the body weight was further reduced, whereas in the group co-administered with stearic acid and pirfenidone (Bleo+P+SA), the body weight was increased compared to the bleomycin administration group, and these results could also be confirmed through the result of quantitatively comparing the body weights on day 21.

Furthermore, as a result of measuring the level of hydroxyproline in order to confirm the amount of collagen accumulated in the tissue, which is commonly used as a major marker of fibrosis, as illustrated in FIG.16B, it was confirmed that compared to the normal control (Ctrl), the level of hydroxyproline was remarkably increased in the case of the bleomycin administration group (Bleo), which induced pulmonary fibrosis, the level of hydroxyproline was partially reduced in the case of the group to which pirfenidone or stearic acid was administered alone, and the level of hydroxyproline was significantly reduced to the level of the normal control in the case of the group to which pirfenidone and stearic acid was co-administered. Further, even through the quantitative analysis results, it could be confirmed that hydroxyproline was inhibited to a very excellent level in the combined administration group (Bleo+Combi)(128%) compared to the pirfenidone single administration group (Bleo+PIR)(105%)

3-2. Anti-Fibrotic Effect by Combined Treatment of Stearic Acid and Nintedanib

To verify the anti-fibrotic effect by the combined treatment of stearic acid and nintedanib, which is another therapeutic agent for idiopathic pulmonary fibrosis, human primary fibroblasts were respectively or simultaneously treated with 5 ng/ml TGF-β, 40 μM stearic acid, and 1.5 or 2 μM nintedanib for 24 hours, and then the expression levels of collagen type 1 (COL-1) and α-SMA, which are markers of fibrosis, were measured, respectively.

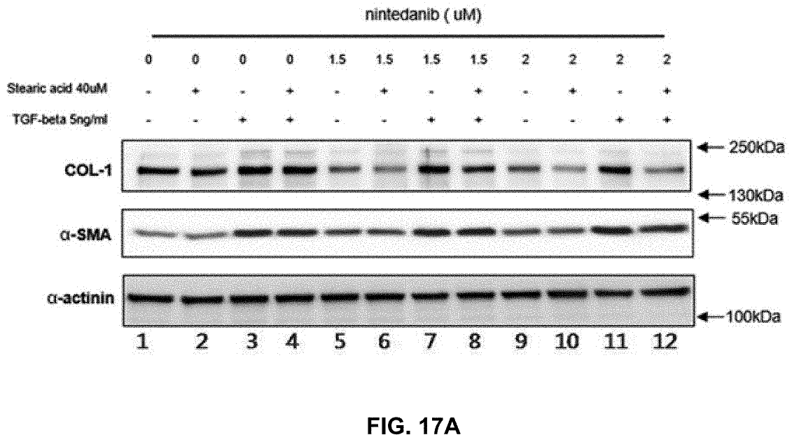

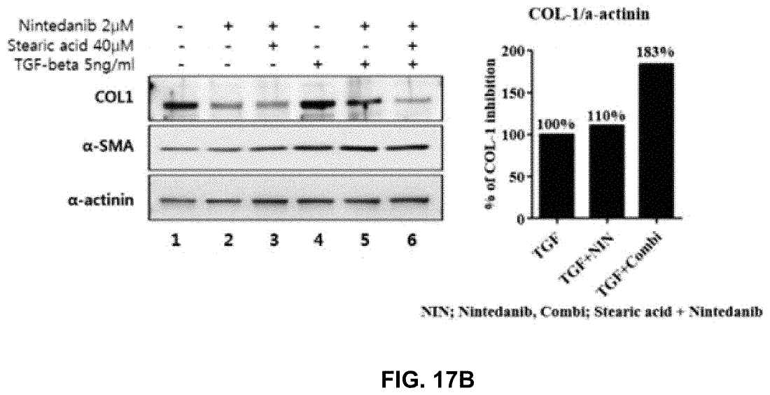

As a result, as illustrated in FIG. 17A, it was observed that when human primary fibroblasts were treated with nintedanib at a concentration of 1.5 μM, COL-1 and α-SMA were not significantly reduced in the combined treatment group with stearic acid (Lane 8) compared to the single treatment group (Lane 7), whereas when human primary fibroblasts were treated with nintedanib at a concentration of 2 μM, COL-1 and α-SMA were reduced in the combined treatment group with stearic acid (Lane 12) compared to the single treatment group (Lane 11),and COL-1 was reduced to a very remarkable level. As can be seen in Lanes 3, 4, 7, and 11, it can be seen that when compared to the TGF-single treatment group (Lane 3), there was no change in expression levels of COL-1 and α-SMA in the stearic acid single treatment group (Lane 4) and the 1.5 and 2 μM nintedanib single treatment groups (Lanes 7 and 11), but when human primary fibroblasts were co-treated with stearic acid and nintedanib, the reduction in a marker of fibrosis was shown, and the higher the concentration of nintedanip was, the greater the anti-fibrotic effect by the combined treatment was.

Referring to the aforementioned results, as can be seen in FIG. 17B, as a result of performing the same experiment and quantitatively analyzing the inhibitory rate against COL-1 only when human primary fibroblasts were treated with 2 μM nintedanib, it can be seen that COL-1 was inhibited very excellently by the combined treatment by confirming that when the expression level of a COL-1 gene caused by TGF was assumed to be 100%, the expression of COL-1 was inhibited to 110% by the nintedanib single treatment (TGF+NIN), whereas the expression of COL-1 was inhibited to 183% during the combined treatment with stearic acid (TGF+Combi).

The above-described description of the present invention is provided for illustrative purposes, and those skilled in the art to which the present invention pertains will understand that the present invention can be easily modified into other specific forms without changing the technical spirit or essential features of the present invention. Therefore, it should be understood that the above-described embodiments are only exemplary in all aspects and are not restrictive.

### INDUSTRIAL APPLICABILITY

According to the present invention, it was confirmed that a more excellent anti-fibrotic effect was exhibited by co-treating stearic acid with a conventional therapeutic agent for pulmonary fibrosis compared to a single treatment with the therapeutic agent. Therefore, it is considered that the co-administration of the aforementioned conventional therapeutic agent for pulmonary fibrosis and stearic acid can enhance the therapeutic effect and ameliorate various drug side effects reported to appear in patients by the therapeutic agent for pulmonary fibrosis, so that the present invention is expected to be usefully used for the treatment of related diseases including idiopathic pulmonary fibrosis.

1. A method for enhancing the sensitivity to a pulmonary fibrosis inhibitor, comprising:
administering to a subject in need thereof an effective amount of the stearic acid, a salt of the stearic acid or a prodrug of the stearic acid as an active ingredient.

2. The method of claim 1, wherein the pulmonary fibrosis inhibitor is selected from the group consisting of pirfenidone, nintedanib, trimethoprim/sulfamethoxazole (co-trimoxazole), a recombinant human pentraxin-2 protein (PRM-151), romilkimab (SAR156597), pamrevlumab, BG00011, treprostinil, TD139, CC-90001, 2-((4(2-ethyl-6-(4-(2-(3-hydroxyazetidin-1-yl)-2-oxoethyl)piperazin-1-yl)-8-methylim idazo[1,2-a]pyridin-3-yl)(methyl)am ino)-4-(4-fluorophenyl)thiazole-5-carbonitrile)(GLPG1690), losartan, tetrathiomolybdate, lebrikizumab, zileuton, nandrolone decanoate, sirolimus, everolimus, vismodegib, fresolimumab, omipalisib (GSK2126458), (3S)-3-[3-(3,5-dimethyl-1H-pyrazol-1-yl)phenyl]-4-{(3S)-3-[2-(5,6,7,8-tetrahydro-1,8-naphthyridin-2-y)ethyl]-1-pyrrolidinyl}butanoic acid (GSK3008348), rituximab, octreotide, 2-[3-[4-(1H-indazol-5-ylamino)-2-quinazolinyl]phenoxy]-N-(1-methylethyl)-acetamide (KD025), tipelukast (MN-001), BBT-877, OLX201, DWN12088, and a salt thereof.

3. The method of claim 1, wherein the pulmonary fibrosis is idiopathic pulmonary fibrosis (IPF).

4. The method of claim 1, wherein the pulmonary fibrosis has an increase in activation of pulmonary fibroblasts and an increase in loss of pulmonary epithelial cells due to TGF-beta compared to the case where there is no pulmonary fibrosis.

5. The method of claim 1, wherein the pulmonary fibrosis has increases in both of fibrosis markers, collagen 1 (COL-1) and α-smooth muscle actin (α-SMA), in pulmonary fibroblasts compared to the case where there is no pulmonary fibrosis.

6. A method for treating pulmonary fibrosis, comprising:
administering to a subject in need thereof an effective amount of (i) stearic acid, a salt of the stearic acid or a prodrug of the stearic acid; and (ii) a pulmonary fibrosis inhibitor.

7. The method of claim 6, wherein the pulmonary fibrosis inhibitor is selected from the group consisting of pirfenidone, nintedanib, trimethoprim/sulfamethoxazole (co-trimoxazole), a recombinant human pentraxin-2 protein (PRM-151), romilkimab (SAR156597), pamrevlumab, BG00011, treprostinil, TD139, CC-90001, 2-((2-ethyl-6-(4-(2-(3-hydroxyazetidin-1-yl)-2-oxoethyl)piperazin-1-yl)-8-methylimidazo[1,2-a]pyridin-3-yl)(methyl)amino)-4-(4-fluorophenyl)thiazole-5-carbonitrile)(GLPG1690), losartan, tetrathiomolybdate, lebrikizumab, zileuton, nandrolone decanoate, sirolimus, everolimus, vismodegib, fresolimumab, omipalisib (GSK2126458), (3S)-3-[3-(3,5-dimethyl-1H-pyrazol-1-yl)phenyl]-4-{(3S)-3-[2-(5,6,7,8-tetrahydro-1,8-naphthyridin-2-yl)ethyl]-1-pyrrolidinyl}butanoic acid (GSK3008348), rituximab, octreotide, 2-[3-[4-(1H-indazol-5-ylamino)-2-quinazolinyl]phenoxy]-N-(1-methylethyl)-acetamide (KD025), tipelukast (MN-001), BBT-877, OLX201, DWN12088, and a salt thereof.

8. The method of claim 7, wherein stearic acid, a salt of the stearic acid, or a prodrug of the stearic acid:pirfenidone are included at a molar concentration ratio of 1:0.5 to 1:25 in the composition.

9. The method of claim 7, wherein stearic acid, a salt of the stearic acid, or a prodrug of the stearic acid:nintedanib are included at a molar concentration ratio of 1:0.01 to 1:5 in the composition.

10. The method of claim 6, wherein the pulmonary fibrosis is idiopathic pulmonary fibrosis (IPF).

11. A method for treating pulmonary fibrosis with resistance to a pulmonary fibrosis inhibitor, comprising:
administering to a subject in need thereof an effective amount of stearic acid, a salt of the stearic acid or a prodrug of the stearic acid as an active ingredient.

12-13. (canceled)
